(19) United States
(12) Patent Application Publication
(10) Pub. No.: US 2026/0347737 A1
(43) Pub. Date: Nov. 5, 2026

(54) A DOCKING STATION FOR A MODULAR INCUBATOR SYSTEM COMPRISING AN IMPROVED GAS DISTRIBUTION SYSTEM

(71) Applicant: ESCO MEDICAL TECHNOLOGIES, UAB, Kaunas (LT)

(72) Inventors: Ricky Lindgaard NIELSEN, Egå (DK); Niels Stengaard HANSEN, Høng (DK)

(21) Appl. No.: 19/124,714

(22) PCT Filed: Jun. 22, 2023
(86) PCT No.: PCT/EP2023/067004

§ 371 Date: Apr. 25, 2025

(30) Foreign Application Priority Data
Nov. 7, 2022 (DK) PA 2022 01010

(51) Int. Cl. C12M 3/00; B01F 35/21; B01F 35/22; C12M 1/00; C12M 1/34; C12M 1/36

(52) U.S. Cl. CPC C12M 23/44; B01F 35/21111; B01F 35/2113; B01F 35/2115; B01F 35/2209; C12M 21/06; C12M 23/40; C12M 41/14; C12M 41/34; C12M 41/40; C12M 41/48

(57) ABSTRACT

A docking station for a modular incubator system includes a number of docking ports for receiving a modular incubator chamber and includes a gas supply system. The docking port includes a docking port outlet opening for gas and a docking port inlet opening for gas. The gas supply system includes a gas source and a gas distribution system. The gas source includes a supply gas outlet and a return gas inlet. The gas distribution system includes a main gas supply line and a main gas return line, and the main gas supply line is fluidly connected to said supply gas outlet of said gas source and the main gas return line is fluidly connected to said return gas inlet of said gas source. The gas distribution system includes a number of manifold pairs, wherein each manifold pair includes an inlet manifold and an outlet manifold.

### FIELD OF THE INVENTION

The present invention generally relates to the field of incubation of viable biological materials and in particular to incubators for IVF (in vitro fertilization) procedures.

More specifically, the present invention relates in a first aspect to a docking station for a modular incubator system.

In a second aspect, the present invention relates to a modular incubator system for incubating a viable biological material comprising the docking station according to the first aspect in combination with one or more modular incubator chambers to be docked in that docking station.

In a third aspect, the present invention relates to a gas source for providing gas to a docking station of a modular incubator docking station for docking a plurality of modular incubator chambers.

In a fourth aspect, the present invention provides a use of a docking station according to the first aspect of the present invention for incubation of a viable biological material.

In a fifth aspect, the present invention provides a use of a modular incubator system according to the second aspect of the present invention for incubation of a viable biological material.

In a sixth aspect, the present invention provides a method of incubating a viable biological material by using a modular incubator system according to the second aspect of the present invention.

### BACKGROUND OF THE INVENTION

The development of in vitro fertilization (IVF) procedures has for the latest few decades resulted in considerably improved methods and techniques which have enhanced success rates of IVF mediated pregnancies and births.

In vitro fertilization involves capturing a ripened egg from a female ovary, fertilizing the ovary with a spermatozoon, incubating the fertilized egg under a controlled environment and subsequently inserting the fertilized and incubated egg in a female's uterus.

As in vitro fertilization is most commonly used by females or couples which notoriously are having problems in getting pregnant the natural way, thus implying some degree of reduced fertility by the male or female counterpart of the couple, or both, and as in vitro fertilization techniques involves quite expensive procedures, these in vitro fertilization techniques are usually performed in a way that seek to optimize efficiency, especially in view of the fact that frequently more than one insertion of a fertilized egg into the female' uterus will be necessary in order to encounter a successful pregnancy.

Additionally, compared to the natural way of getting pregnant, in respect of a couple wherein an individual is having a generic disease or in case of suspicion of such disease, an IVF mediated pregnancy may be advantageous.

Accordingly, in order to make the in vitro fertilization techniques efficient, the female is typically subjected to a hormone treatment prior to harvesting eggs from her ovary. Such hormone treatment will make the female ovary ovulate not only one egg, but a multitude of eggs at the same time.

In order to increase the chance of a viable and successful pregnancy more than one egg from the same female will accordingly be fertilized and incubated concurrently in an incubator.

Prior art incubators include a compartment which allows for accommodating more than one culture dish comprising the fertilized eggs.

Performing a successful in vitro fertilization and incubation of a fertilized egg is not an easy task. One of the major reasons for the rather low success rate of in vitro fertilizations is the absence of reliable methods for providing and maintaining optimum incubation conditions for the embryo.

Some improved prior art incubators comprise a housing having one or more doors for providing access to the interior of the incubator. The interior of the incubator holds one or more culture dishes accommodating the embryos to be cultured. Such incubators may be provided with various regulation means for controlling humidity, temperature and gas composition of the interior of the incubator.

Recently, smaller modular incubators have been introduced in the marked. These modular incubators are configured to be stored in docking ports in a docking station which may provide the controlling of the physical and chemical parameters to be encountered by the embryos being accommodated therein. Once any manual manipulation steps in respect of the embryo is needed, such as manual inspection or adding or removing or exchanging growth media, the modular incubator will be removed from the docking station and arranged on a laboratory bench for easy access to the embryo.

These modular incubators and the corresponding docking ports of the docking station may even be provided with gas connectors so that once a modular incubator is docked in a docking port of the docking station, gas connectors of the modular incubator and corresponding gas connectors of the docking port engage in such a way that a gas having a desired gas composition can be conveyed from the docking station through each modular incubator and back to the docking station via these gas connectors. In this way a desired gas composition can delivered to the interior of the modular incubators in the period of time where a modular incubator is being docked in the docking port of the docking station.

The gas composition is typically controlled by use of a gas mixing box enabling addition of CO₂ and N₂. An outlet of the gas mixing box is fluidly connected to an inlet gas connector of each modular incubator and a return inlet of the gas mixing box is fluidly connected to an outlet gas connector of each modular incubator. Accordingly, gas is circulated through the modular incubator and through the gas mixing box. Upon controlling the gas composition to be supplied to the incubators, CO₂ is added to the gas mixing box in order to reach a desired CO₂ concentration and N₂ is being added to the gas mixing box in order to lower the O₂ level to a desired concentration. As leaks in the gas supply system is inevitably, atmospheric air will find its way into the system and therefore oxygen will never be depleted below the desired oxygen concentration.

By constantly monitoring the CO₂ concentration and the O₂ concentration of the gas circulating in the system and by constantly regulating the gas composition leaving the gas mixing box, in response to any deviations from the desired and predetermined gas composition, it possible to ensure that the gas leaving the gas mixing box to be supplied to the modular incubators is having the desired and predetermined optimum composition.

It has been found that in respect of gas composition in the interior of an incubator chamber, even small deviations from what is considered to be an optimum gas composition, may have detrimental effects on the quality of the biological material being incubated therein, and hence ultimately also of the success rate of a resulting pregnancy in case of incubating an embryo.

For this reason, it is important that each of the modular incubators, to the extent possible, constantly contain the same gas composition which is a predetermined gas composition that is considered optimum for incubation of an embryo.

It has been found that even if the optimum gas composition is being supplied in the supply lines leading to and from each of the modular incubators, it may happen that the gas composition varies for an undesirably long time in the interior of the various modular incubators when going from one modular incubator to another in a non-steady state; that is, where one or more modular incubators is/are has/have been shortly removed from the docking station for performing visual inspection and manual replenishing, removal or exchange of growth medium to the biological materials being incubated, thereby letting in atmospheric air in the interior of the modular incubator.

The same applies in a situation where the desired gas composition, being mixed in the gas mixing box, is deliberately varied dynamically over time and supplied to the modular incubators.

The inventors expect that these problems may originate from an improper design of the gas supply system supplying gas between the gas mixing box and the modular incubators and that variations of the gas composition, between the incubator chambers, may originate from variations in pressure drop through the interior of a modular incubator chamber of the modular incubator system between the inlet and outlet openings for gas in the docking ports of the docking station.

It has also been found that the latency or sluggishness in arriving at a steady state situation in the gas supply system after having removed and returned a modular incubator chamber makes it very difficult to regulate the supply of CO₂ and N₂ gas to the gas mixing box in order to arrive at a steady state situation of constant and the desired gas composition in the whole supply system, supplying gas to the modular incubators.

Prolonged deviations of the gas composition from what is considered to be an optimum gas composition may accordingly and ultimately represent enhanced risks that the IVF procedure ends in an unsuccessful pregnancy once the embryo has been inserted into a female's uterus.

Therefore, in order to secure optimum incubation conditions of the embryos being incubated in the modular incubators it is of paramount importance that the gas composition of gas being present in the interior of each modular incubator chamber deviates as little as possible from that particular gas composition, which is considered the optimum composition, and preferably is varies as little as possible when going from one modular incubator to another.

Accordingly, a need persists for an improved docking station and associated modular incubator system which ensure minimizing variations from a desired and/or optimum gas composition in the interior of a plurality modular incubators being docked in docking ports of a docking station when going from one modular incubator to another.

It is an objective of the present invention to fulfill such need.

### BRIEF DESCRIPTION OF THE INVENTION

This objective is fulfilled according to the present invention in its various aspects.

Accordingly, the present invention relates in a first aspect to a docking station for a modular incubator system, wherein said docking station comprises:
a number of docking ports for receiving a modular incubator chamber; and
gas supply system;
wherein in respect of one or more of said docking ports, preferably in respect of all said docking ports of said docking station, said docking port comprises a docking port outlet opening for gas and a docking port inlet opening for gas;
wherein said gas supply system comprises a gas source and a gas distribution system;
wherein said gas source comprises a supply gas outlet and a return gas inlet;
wherein said gas distribution system comprises a main gas supply line and a main gas return line;
wherein said main gas supply line of said gas distribution system is being fluidly connected to said supply gas outlet of said gas source and wherein said main gas return line is being fluidly connected to said return gas inlet of said gas source;
wherein said gas distribution system comprises a number of manifold pairs, wherein each manifold pair comprises an inlet manifold and an outlet manifold;
wherein said inlet manifold of each manifold pair is fluidly connected to said main gas supply line at an inlet manifold connection point;
wherein said outlet manifold of each manifold pair is fluidly connected to said main gas return line at an outlet manifold connection point;
wherein a gas supply line reference point arranged at a position upstream in relation to inlet manifold connection points of all said inlet manifolds is being defined on said main gas supply line;
wherein a gas return line reference point arranged at a position downstream in relation to outlet manifold connection points of all said outlet manifolds is being defined on said main gas return line;
wherein each manifold pair is connected to one or more docking ports of said docking station in such a way that in respect of a specific manifold pair, and in respect of said one or more docking ports being connected thereto, said docking port outlet opening for gas of said docking port is being fluidly connected to said inlet manifold, and said docking port inlet opening for gas of said docking port is being fluidly connected to said outlet manifold;
wherein said gas distribution system is designed in such a way that in respect of two or more docking ports of said docking station, preferably in respect of all docking ports of said docking station, the travel distance D for gas to travel from said gas supply line reference point to said docking port and from said docking port to said gas return line reference point upon conveying gas to and from said docking port is being essentially equal; wherein the travel distance D, in respect of a specific docking port, is defined as:

D=D1+D2+D3+D4;
wherein D1 is defined as the distance from said gas supply line reference point to the corresponding inlet manifold connection point at said main gas supply line;
wherein D2 is defined as the distance from said corresponding manifold connection point at said main gas supply line to said docking port outlet opening for gas;
wherein D3 is defined as the distance from said docking port inlet opening for gas to the corresponding outlet manifold connection point at said main gas return line;
wherein D4 is defined as the distance from said outlet manifold connection point at said main gas return line to said gas return line reference point.

In a second aspect the present invention relates to a modular incubator system comprising a docking station according to the first aspect of the present invention in combination with one or more modular incubator chambers; wherein in respect of one or more of said one or more modular incubator chambers, said modular incubator chamber comprises:
a housing having a first end and a second end, thereby defining a longitudinal direction X between said first end and said second end;
wherein said housing comprises a lid, wherein said lid is being configured to be able to shift between an open configuration allowing access to the interior of said modular incubator chamber and a closed configuration, sealing off access to the interior of said modular incubator chamber;
wherein said modular incubator chamber, at said interior thereof, comprises a culture dish support for positioning a culture dish with the view to accommodate one or more biological materials M within the housing of said modular incubator chamber;
wherein said modular incubator chamber comprises a chamber inlet opening for gas and a chamber outlet opening for gas, wherein said chamber inlet opening for gas and said chamber outlet opening for gas are being in fluid connection with the interior of said modular incubator chamber.

In a third aspect, the present invention relates to a gas source for providing gas to a docking station, wherein said gas source comprises a supply gas outlet to be connected to a main gas supply line of a docking station, and wherein said gas source comprises a return gas inlet to be connected to a main gas return line of said docking station; wherein said gas source is as defined in respect of the first aspect of the present invention.

In a fourth aspect, the present invention provides a use of a docking station according to the first aspect of the present invention for incubation of a viable biological material.

In a fifth aspect, the present invention provides a use of a modular incubator system according to the second aspect of the present invention for incubation of a viable biological material.

In a sixth aspect, the present invention provides a method of incubating a viable biological material, wherein said method comprises:
i) providing a modular incubator system according to the first aspect of the present invention;
ii) providing a viable biological material;
iii) arranging said viable biological material in a culture dish and subsequently arranging said culture dish in the interior of a modular incubator chamber of said modular incubator system;
iv) docking said modular incubator chamber in a docking port of said docking station of said modular incubator system;
v) allowing said viable biological material to be incubated in said modular incubator chamber;
vi) via said gas supply system of said docking station, supplying gas through the interior of said modular incubator chamber of said modular incubator system.

The present invention in its various aspects ensures that in respect of the various docking ports of the docking station an equal gas flow is encountered through all the modular incubator chambers being docking therein, and this has turned out to reduce the latency or sluggishness of returning to a steady state situation in respect of gas composition after having deviated from a steady state situation, for example after having shortly removed and returned a modular incubator chamber from and to its docking port with the view to perform visual inspection and/or to perform manual replenishing, removal or exchange of growth medium in respect of the biological materials being incubated therein.

### BRIEF DESCRIPTION OF THE FIGURES

FIG. 1 is a perspective view illustrating the concept of a modular incubator system of the present invention comprising a docking station with a plurality of docking ports and a plurality of modular incubator chambers to be docked in the docking ports of the docking station.

FIG. 2 is a perspective view of a modular incubator chamber of the modular docking system of FIG. 1.

FIG. 3 is a plan view of the modular incubator chamber illustrated in FIG. 2 as seen from above.

FIG. 4 is a plan view of the modular incubator chamber illustrated in FIGS. 2 and 3 as seen from a rear side.

FIG. 5 is a cross-sectional view of a modular incubator chamber of the modular incubator system of the invention.

FIGS. 6a and 6b illustrate the working modes of the valves of a valve system of the modular incubator chamber and the associated docking port of the docking station of the docking system of the present invention.

FIG. 7 is a diagram illustrating the concept of the gas supply system which in incorporated in the docking station of the modular incubator system of the present invention.

FIG. 8 is a diagram illustrating details of the gas distribution system of the gas supply system of the docking station of the present invention.

FIG. 9 is a diagram illustrating one embodiment of a design of a gas supply system comprising a gas source to be used with the docking station of the present invention.

FIG. 10 is a diagram illustrating the working mode of the controlling of the modular incubator system according to the invention.

DETAILED DESCRIPTION OF THE INVENTION

### The First Aspect of the Present Invention

The present invention relates in its first aspect to docking station 400 for a modular incubator system 500, wherein said docking station comprises:
a number of docking ports 402 for receiving a modular incubator chamber 300; and
gas supply system 200;
wherein in respect of one or more of said docking ports 402, preferably in respect of all said docking ports of said docking station 400, said docking port comprises a docking port outlet opening for gas 404 and a docking port inlet opening for gas 406;
wherein said gas supply system 200 comprises a gas source 202 and a gas distribution system 204;

wherein said gas source 202 comprises a supply gas outlet 206 and a return gas inlet 208; wherein said gas distribution system 204 comprises a main gas supply line 210 and a main gas return line 212;
wherein said main gas supply line 210 of said gas distribution system 204 is being fluidly connected to said supply gas outlet 206 of said gas source 202 and wherein said main gas return line 212 is being fluidly connected to said return gas inlet 208 of said gas source 202;
wherein said gas distribution system 204 comprises a number of manifold pairs 214, wherein each manifold pair comprises an inlet manifold 216 and an outlet manifold 218;
wherein said inlet manifold 216 of each manifold pair 214 is fluidly connected to said main gas supply line 210 at an inlet manifold connection point 220;
wherein said outlet manifold 218 of each manifold pair 214 is fluidly connected to said main gas return line 212 at an outlet manifold connection point 222;
wherein a gas supply line reference point 224 arranged at a position upstream in relation to inlet manifold connection points 220 of all said inlet manifolds 216 is being defined on said main gas supply line 210;
wherein a gas return line reference point 226 arranged at a position downstream in relation to outlet manifold connection points 222 of all said outlet manifolds 218 is being defined on said main gas return line 212;
wherein each manifold pair 214 is connected to one or more docking ports 402 of said docking station 400 in such a way that in respect of a specific manifold pair 214, and in respect of said one or more docking ports 402 being connected thereto, said docking port outlet opening for gas 404 of said docking port 402 is being fluidly connected to said inlet manifold 216, and said docking port inlet opening for gas 406 of said docking port 402 is being fluidly connected to said outlet manifold 218;
wherein said gas distribution system 204 is designed in such a way that in respect of two or more docking ports 402 of said docking station, preferably in respect of all docking ports 402 of said docking station, the travel distance D for gas to travel from said gas supply line reference point 224 to said docking port 402 and from said docking port 402 to said gas return line reference point 226 upon conveying gas to and from said docking port 402 is being essentially equal; wherein the travel distance D, in respect of a specific docking port 402, is defined as:

D=D1 +D2 +D3 +D4;
wherein D1 is defined as the distance from said gas supply line reference point 224 to the corresponding inlet manifold connection point 220 at said main gas supply line 210;
wherein D2 is defined as the distance from said corresponding manifold connection point 220 at said main gas supply line 210 to said docking port outlet opening for gas 404;
wherein D3 is defined as the distance from said docking port inlet opening for gas 406 to the corresponding outlet manifold connection point 222 at said main gas return line 212;
wherein D4 is defined as the distance from said outlet manifold connection point 222 at said main gas return line 212 to said gas return line reference point 226.

As mentioned above, the design of the gas distribution system of the docking station ensures that in respect of the various docking ports an equal gas flow can be encountered through all the modular incubator chambers being docking therein.

Such equal gas flow reduces the latency or sluggishness of returning to a steady state situation in respect of gas composition after having deviated from a steady state situation, for example after shortly having removed and returned a modular incubator chamber from and to its docking port with the view to perform visual inspection and perform manual replenishing, removal or exchange of growth medium in respect of the biological materials being incubated therein.

Accordingly, with the docking station of the first aspect of the present invention, the period of time in which the gas composition deviates from what is considered optimum and desirable will be reduced. This ultimately implies higher quality incubations of biological materials, such as oocytes or embryos.

In one embodiment of the docking station according to the first aspect of the present invention, the distance D in respect of one docking port 402 is having a magnitude which is greater than the distance D in respect of another docking port 402, preferably in respect of any other docking port by 10 % or less, such as 9 % or less, e.g. 8 % or less, for example 7 % or less, such as 6 % or less, e.g. 5 % or less,, such as 4 % or less, for example 3 % or less, such as 2 % or less, or 1 % or less.

Here is accordingly defined what may be understood by the term “the travel distance D for gas to travel from said gas supply line reference point to said docking port and from said docking port to said gas return line reference point upon conveying gas to and from said docking port is being essentially equal”.

In one embodiment of the docking station according to the first aspect of the present invention the docking ports 402 are grouped into one or more groups 228 of docking ports 402 wherein, in respect of each docking port 402 belonging to a specific group 228 of docking ports 402, said docking port outlet opening for gas 404 is fluidly connected to the same inlet manifold 216; and wherein in respect of each docking port 402 belonging to a specific group 228 of docking ports 402, said docking port inlet opening for gas 406 is fluidly connected to the same outlet manifold 218.

In one embodiment of this embodiment, the number of groups 228 of docking ports 402 is selected from the ranges 1-20 or more, such as 2-19, for example 3-18, such as 4-17, such as 5-16, e.g. 6-15, such as 7-14, e.g. 8-13, such as 9-12 or 10-11.

In one embodiment of the docking station according to the first aspect of the present invention the number of docking ports 402 in each group 228 of docking ports 402 independently is being selected from the ranges 2-25 or more, such as 3-24, for example 4-23, e.g. 5-22,such as 6-21, e.g. 7-20, for example 8-19, such as 9-18, for example 10-17, such as 11-16, e.g. 12-15 or 13-14.

In one embodiment of the docking station according to the first aspect of the present invention the number of groups 228 of docking ports 402 is two or more, and each group of docking ports is arranged as a shelf, wherein said shelves of groups 228 of docking ports 402 are being arranged on top of each other.

Accordingly, grouping of the docking ports 402 in this way enables arranging the docking ports into shelves arranged on top of each other. In theory there are no limits of the number of docking ports 402 which may be included in a docking station 400.

In one embodiment of the docking station according to the first aspect of the present invention the main gas supply line 210 is provided with a main line extension connector 240a for enabling extension of said main gas supply line 210, and wherein said main gas return line 212 is provided with a main line extension connector 240b for enabling extension of said main gas return line 212 with the view to add an extension of the main gas supply line and an extension of the main gas return line, and thereby enable adding one or more groups 228 of docking ports 402, each being fluidly connected to an added inlet manifold 216 and fluidly connected to an added outlet manifold 218, wherein said added inlet manifold 216 is fluidly connected to said added extension of said main gas return line 210, and wherein said added outlet manifold 218 is fluidly connected to said added extension of said main gas return line 212.

Hereby the docking station may be supplied by the manufacturer as a modular docking station which itself may be extended by extension of the main gas supply line and an extension of the main gas return line.

In one embodiment of this embodiment the extension connector 240a is arranged at said main gas supply line 210 at a position downstream in relation to all inlet manifolds, and wherein said extension connector 240b is arranged at said return gas supply line 212 at a position downstream in relation to all outlet manifolds; or wherein said extension connector 240a is arranged at said main gas supply line 210 at a position upstream in relation to all inlet manifolds, and wherein said extension connector 240b is arranged at said return gas supply line 212 at a position upstream in relation to all outlet manifolds; or wherein said extension connectors 240a,240b are arranged at said main gas supply line 210 and said return gas supply line 212 at a position therebetween.

In one embodiment of the docking station according to the first aspect of the present invention the docking station is being prepared for expansion by addition of one or more additional and new groups 228a of docking ports 402, wherein each such new group 228a of docking ports 402 comprises a new inlet manifold 216 comprising an inlet manifold connector 230, and a new outlet manifold 218 comprising an outlet manifold connector 232; wherein said main gas supply line 210 of said gas distribution system 204 comprising one or more connectors 234 which is/are being adapted to be connected to said inlet manifold connector 230 of said new inlet manifold 216; and wherein said main gas return line 212 of said gas distribution system 204 comprises one or more connectors 236 which is/are being adapted to be connected to said outlet manifold connector 232 of said new outlet manifold 218; wherein each new group 228a of docking ports comprises a number of new docking ports 402, wherein in respect of one or more of said new docking ports 402, said new docking port 402 comprises a docking port outlet opening for gas 404 which is being fluidly connected to said new inlet manifold 216; and wherein in respect of one or more of said new docking ports 402, said new docking port comprises a docking port inlet opening for gas 406 which is being fluidly connected to said new outlet manifold 218; thereby enabling expansion of said docking station 400 with new groups 228a of docking ports 402.

This embodiment represents an alternative way of expanding an existing docking station by enabling addition of new groups 228 of docking ports.

In one embodiment of the docking station according to the first aspect of the present invention and in respect of one or more of said docking ports 402, preferably in respect of all said docking ports, said docking port outlet opening for gas 404 comprises a valve 4, and said docking port inlet opening for gas 406 comprises a valve 4; wherein said valve 4 of said docking port outlet opening for gas 404 and said valve 4 of said docking port inlet opening for gas 406 each comprises a valve body 16 having a front end 20, a rear end 22 and a through-going channel 24 therein, and a spring-loaded displaceable valve element 18, wherein said displaceable valve element 18 is being arranged in said through-going channel 24; wherein said displaceable valve element 18 is being configured to be displaceable in said through-going channel 24 of said valve body 16 in such a way, that when not acted upon by an external force, said spring-loaded displaceable valve element 18 is not being displaced in said through-going channel 24 of said valve body 16, thereby making said valve attain a closed configuration blocking passage of gas through said through-going channel 24, and in such a way, that when acted upon by an external force, said spring-loaded displaceable valve element 18 is being displaced in said through-going channel 24 of said valve body 16, thereby making said valve 4 attain an open configuration, allowing passage of gas through said through-going channel 24.

Hereby is ensured that gas will not escape into the area of the docking port 402 through the docking port outlet opening for gas 404 and the docking port inlet opening for gas 406, unless a modular incubator chamber 300 is being docked in that docking port 402.

In one embodiment of the docking station according to the first aspect of the present invention the gas distribution system 204 comprises one or more shunts 238 fluidly connecting said main gas supply line 210 with said main gas return line 212, thereby enabling circulation of gas in said gas distribution system in a situation where no modular incubator chamber 300 is being docked in a docking port 402 of said docking station 400.

Inclusion of such shunt(s) will ensure continued circulation of gas through the gas distribution system 204 of the docking station, in case no modular incubator chamber 300 is being docked in a docking port 402 and thereby also enable constantly performing regulation of the gas composition with the view to ensure that the gas composition is optimum and desired, once a modular incubator chamber 300 is being docked in a docking port 402 of the docking station 400.

In one embodiment of this embodiment the one or more of the shunts 238 fluidly connect(s) an outlet manifold 218 to said main gas supply line 210; or wherein one or more of said shunts 238 fluidly connects an inlet manifold 216 to said main gas return line 212; or wherein one or more of said shunts 238 fluidly connects an inlet manifold 216 to an outlet manifold 218.

In another embodiment and in respect of one or more of said shunts, said shunt is arranged in such a way that the travel distance for gas from said gas supply line reference point 224 to said shunt plus the travel distance from said shunt to said gas return line reference point 226 is essentially equal to the distance D.

In one embodiment of the docking station according to the first aspect of the present invention and in respect of two or more of said docking ports 402, preferably in respect of all said docking ports 402, the internal parts of said docking ports 402, in terms of dimensions and geometry, are essential identical.

Such design will further reduce fluctuations in the gas flow flowing in the gas supply system, when going from one docking port 402 to another.

In one embodiment of the docking station according to the first aspect of the present invention and in respect of two or more of said docking ports 402, preferably in respect of all said docking ports 402, the docking port outlet opening for gas 404 comprises a flow restrictor for restricting the magnitude of flow of gas flowing into said docking port 402.

In one embodiment of the docking station according to the first aspect of the present invention the flow restrictor comprises a tube through which the gas is conveyed to said docking port 402, wherein said tube optionally is having a cross-sectional area selected from the ranges of 0.2-8 mm², such as 0.5-7 mm², for example 1-6 mm², such as 2-5 mm² or 3-4 mm²; and/or the length of said tube is optionally selected from the ranges of 5-30 mm, such as 8-25 mm, for example 10-22 mm, e.g. 15-20 mm.

Such a flow restrictor aids in balancing the flow of gas through the docking ports 402 comprising a modular incubator chamber 300 with the capacity of the gas supply system 200 and thereby also aids in making the flow of gas through the different docking ports 402 equal to each other.

In one embodiment of the docking station according to the first aspect of the present invention and in respect of one or more docking ports 402 of said docking station 400, said docking port comprises an image capturing device 408.

Inclusion of an image capturing device 408 allows capturing images of a biological material M being accommodated in the interior 306 of a modular incubator chamber 300, once being docked in said docking port 402.

In one embodiment of this embodiment and in respect of one or more specific docking ports 402 of said docking station 400, said specific docking port comprises its own dedicated image capturing device 408 which is configured to only capture images relating to a modular incubator chamber 300 which is being docked in said specific docking port 402.

In another embodiment of the above embodiment and in respect of a number N of adjacently arranged docking ports 402 of said docking station 400, said adjacently arranged docking ports share a common image capturing device 408 in the sense that one and only one image capturing device is responsible for capturing images relating to a modular incubator chamber 300 which is being docked in one of said N adjacently arranged docking ports 402, wherein said docking station comprises a displacement device 482, such as an electrically driven and remotely controlled displacement device 482 for enabling displacement of said common image capturing device 408 in relation to said N adjacently arranged docking ports 402 of said docking station 400.

Hereby one image capturing device is responsible for the capturing of images of biological materials being accommodated in different modular incubator chambers which are being docked in different docking port 402 of the docking station 400.

In one embodiment said number N is being an integer selected in the ranges of 2-25 or more, such as 4-22, for example 6-20, such as 8-18, such as 10-16 or 12-14.

Independently, one or more image capturing devices 408, preferably all image capturing devices 408 of the docking station 400 may comprise or be coupled to a displacement device 482, such as an electrically driven and remotely controlled displacement device 482 for enabling displacement of said common image capturing device 408 in a direction transversal to the longitudinal direction X of a modular incubator chamber 300 being docked in a docking port 402 with the view to enable such capturing device 408 to focus on more than one culture well in a culture dish 310 being accommodated in the interior of the modular incubator chamber 300, wherein such culture wells are arranged in such direction transversal to the longitudinal direction X.

In one embodiment of the docking station according to the first aspect of the present invention, the one or more of said image capturing devices 408 of said docking ports 402 comprise(s) microscopic optics so as to enable capturing of microscope images.

Hereby magnified images may be captured which improves study of the morphological nature of the biological materials being incubated.

In one embodiment of the docking station according to the first aspect of the present invention, the gas source 202 of said gas supply system 200 comprises a gas mixing box 242 comprising said supply gas outlet 206 and said return gas inlet 208 of said gas source, wherein said main gas supply line 210 of said gas distribution system 204 is being fluidly connected to said supply gas outlet 206, and wherein said main gas return line 212 of said gas distribution system 204 is being fluidly connected to said return gas inlet 208 of said gas source 202, thereby forming a flow loop 244 comprising said gas distribution system 204 and said gas mixing box 242; wherein said flow loop comprises a pump 246.

Hereby circulating gas in said loop, and also through the gas distribution system of the docking station is possible.

The purpose of the gas source is to provide and deliver a desired gas composition to the gas distribution system 204, including the various docking ports 402 of the docking station 400.

In one embodiment of this embodiment the pump 246 is being arranged downstream in relation to said main gas return line 212.

In one embodiment the flow loop 244 comprises a pump oscillation damper 247, wherein said pump oscillation damper optionally is being arranged immediately downstream in relation to said pump 246.

The pump oscillation damper will equalize small and rapid pressure variations caused by each pump stroke of the pump.

In one embodiment of the docking station according to the first aspect of the present invention, the flow loop 244 comprises a pressure sensor, such as a differential pressure sensor 248 for sensing the pressure of gas supplied to said main gas supply line 210 of said gas distribution system 204, wherein said pressure senor 248 optionally is being arranged immediately upstream in relation to said main gas supply line 210 of said gas distribution system 204.

The pressure sensor 248 allows for regulating the pump 246 on order to maintain a desired pressure in the flow loop 244.

In one embodiment the pressure sensor 248 is being a differential pressure sensor, sensing a pressure relative to the pressure of the return gas inlet 208.

In one embodiment the flow loop 244 comprises a release valve 249 for enabling pressure relief in said flow loop, wherein said release valve optionally is being arranged immediately downstream in relation to said main gas return line 212 of said gas distribution system 402.

The pressure release valve 249 enables improved control of the pressure in the flow loop 344. In one embodiment of the docking station according to the first aspect of the present invention, the gas mixing box 242 comprises an inlet for N₂ gas 250; and an inlet for CO₂ gas 251, wherein said inlet for N₂ gas 250 is fluidly connected to an N₂ valve 252 for regulating the inflow of N₂, and an N₂ mass flow sensor 253 arranged downstream of said N₂ valve 252 for sensing the amount of N₂ flowing into said gas mixing box 242; and wherein said inlet for CO₂ gas 251 is fluidly connected to a CO₂ valve 254 for regulating the inflow of CO₂, and an CO₂ mass flow sensor 255 arranged downstream of said CO₂ valve 254 for sensing the amount of CO₂ flowing into said gas mixing box 242.

Herby it is possible to control the inlet of N₂ gas and the inlet of CO₂ gas into the gas mixing box 242 with the view to obtain a desired, predetermined and optimum gas composition in the gas mixing box 242.

In one embodiment of the docking station according to the first aspect of the present invention, the flow loop 244 comprises a mass flow sensor 256 arranged at an upstream position in relation to said gas mixing box 242 for sensing the amount of return gas entering said gas mixing box.

Information relating to the amount of return gas entering said gas mixing box is used for determining the total amount of N₂ gas and CO₂ gas which needs to be introduced into the gas mixing box 242.

In one embodiment of the docking station according to the first aspect of the present invention, the gas source 202 comprises an O₂ sensor 258 for sensing the concentration of O₂ exiting said gas distribution system 204; and wherein said gas source 202 comprises a CO₂ sensor 260 for sensing the concentration of CO₂ exiting said gas distribution system 204, wherein said O₂ sensor and/or said CO₂ sensor optionally is/are being arranged downstream in relation to said pump 246.

Information relating to the concentration of O₂ and the concentration of CO₂ exiting said gas distribution system 204 is used for determining the specific amount of N₂ gas and the specific amount of CO₂ gas which needs to be introduced into the gas mixing box 242.

In one embodiment of the docking station according to the first aspect of the present invention, the gas source 202 comprises a temperature sensor 262 for sensing the temperature of gas circulating in said flow loop 244, wherein said temperature sensor optionally is being arranged downstream in relation to said pump 246, preferably at a position corresponding to the position of said O₂ sensor 258.

In one embodiment of the docking station according to the first aspect of the present invention, the gas source 202 comprises a pressure sensor 264 for sensing the absolute pressure in said flow loop 244 wherein said pressure sensor optionally is being arranged downstream in relation to said pump 246, preferably at a position corresponding to the position of said CO₂ sensor 260.

The temperature sensor 262 and pressure sensor 264 are useful for performing compensation of the readings of the O₂ sensor 258 due to temperature sensitivity thereof and the readings of the CO₂ sensor 260 due to sensitivity thereof towards pressure.

In one embodiment of the docking station according to the first aspect of the present invention, the flow loop 244 comprises a UV sanitizer 266 for sanitizing gas flowing in said flow loop 244 via electromagnetic radiation in the UV range, wherein said UV sanitizer optionally being arranged immediately downstream in relation to said main gas return line 212.

In one embodiment of the docking station according to the first aspect of the present invention, the gas source 202 comprises one or more filters 268, such as HEPA and/or VOCs filters, wherein such a filter is being arranged immediately upstream in relation to said main gas supply line 210, and/or wherein such a filter is being arranged immediately upstream in relation to the inlet for N₂ gas 250 into said gas mixing box 242; and/or wherein such a filter is being arranged immediately upstream in relation to the inlet for CO₂ gas 251 into said gas mixing box 242.

In one embodiment of the docking station according to the first aspect of the present invention, the gas source 202 comprises a gas mixing control system 270, wherein said gas mixing control system is electrically connected to one or more of the following sensors for receiving sensing signals therefrom: said N₂ mass flow sensor 253 for sensing the amount of N₂ flowing into said gas mixing box; said CO₂ mass flow sensor 255 for sensing the amount of CO₂ flowing into said gas mixing box; said mass flow sensor 256 for sensing the amount of return gas entering said gas mixing box; said O₂ sensor 258 for sensing the concentration of O₂ exiting said main gas return line 212 of said gas distribution system 204; said CO₂ sensor 260 for sensing the concentration of CO₂ exiting said main gas return line 212 of said gas distribution system 204; said temperature sensor 262 for sensing the temperature circulating in said flow loop 244; said pressure sensor 264 for sensing an absolute pressure in said flow loop 244, said pressure sensor 248 for sensing the pressure of gas supplied to said gas main gas supply line 210 of said distribution system 204.

This embodiment enables gaining information of various parameters which are to be used in providing a feed back when controlling the operation of the gas source 202.

In one embodiment of the docking station according to the first aspect of the present invention, the gas mixing control system 270 is electrically connected to one or more of the following elements for control thereof: said N₂ valve 252 for regulating the inflow of N₂ into said gas mixing box 242; said CO₂ valve 254 for regulating the inflow of CO₂ to said gas mixing box 242; said pump 246 for circulating gas in said flow loop 244; said release valve 249.

This embodiment enables providing a feed back when controlling the operation of the gas source 202.

In one embodiment the gas mixing control system 270 is being configured to receive input from said pressure sensor 248 and on the basis thereof control said pump 246, optionally also to activate said release valve 249 in order to maintain a desired and predetermined pressure of gas supplied to said main gas supply line 210 of said gas distribution system 204.

Hereby the pressure in the flow loop 244 can be controlled.

In one embodiment of the docking station according to the first aspect of the present invention, the gas mixing control system 270 is being configured to receive input from said mass flow sensor 256, and on the basis on said input to determine the total amount of CO₂ gas and N₂ gas needed to be supplied via said inlet for CO₂ gas 251 and via said inlet for N₂ gas 250 according to desired and predetermined criteria.

In one embodiment of the docking station according to the first aspect of the present invention, the gas mixing control system 270 is being configured to receive input from said CO₂ sensor 260 and said O₂ sensor 258, and on the basis of the CO₂ concentration sensed, is configured to control said CO₂ valve 254, by transmitting a control signal thereto, and thereby regulating the inflow of CO₂ gas in order to reach a desired and predetermined CO₂ concentration, and wherein subsequently, said gas mixing control system 270 on the basis of the O₂ concentration sensed, is configured to control said N₂ valve 252, by transmitting a control signal thereto, and thereby regulating the inflow of N₂ gas in order to reach a desired and predetermined O₂ concentration.

In one embodiment of the docking station according to the first aspect of the present invention, the gas mixing control system 270 is configured to use the input from said temperature sensor 262 for compensating the temperature sensitivity of said O₂ sensor 258.

In one embodiment of the docking station according to the first aspect of the present invention, the gas mixing control system 270 is configured to use the input from said pressure sensor 264 for compensating the pressure sensitivity of said CO₂ sensor 260.

In one embodiment of the docking station according to the first aspect of the present invention, the gas mixing control system 270 is being configured to maintain a pressure of gas supplied to said main gas supply line 210 of said gas distribution system 204, relative to the ambient atmospheric pressure, of 3-20 mbar, such as 5-18 mbar, such as 10-15 mbar above that ambient atmospheric pressure.

In one embodiment of the docking station according to the first aspect of the present invention, the gas mixing control system 270 is being configured to maintain a CO₂ concentration of gas entering said main gas supply line 210 of said gas distribution system 204 in the range of 5-10%, such as 6-9 % or 7-8 %; and/or an O₂ concentration of gas entering said main gas supply line 210 of said gas distribution system 204 in the range of 5-10%, such as 6-9% or 7-8%.

### The Second Aspect of the Present Invention

In the second aspect the present invention relates to a modular incubator system 500 comprising a docking station 400 according to the first aspect of the present invention in combination with one or more modular incubator chambers 300; wherein in respect of one or more of said one or more modular incubator chambers 300, said modular incubator chamber 300 comprises: a housing 302 having a first end 340 and a second end 342, thereby defining a longitudinal direction X between said first end and said second end; wherein said housing comprises a lid 304, wherein said lid is being configured to be able to shift between an open configuration allowing access to the interior 306 of said modular incubator chamber and a closed configuration, sealing off access to the interior of said modular incubator chamber; wherein said modular incubator chamber 300, at said interior 306 thereof, comprises a culture dish support 308 for positioning a culture dish 310 with the view to accommodate one or more biological materials M within the housing 302 of said modular incubator chamber 300; wherein said modular incubator chamber 300 comprises a chamber inlet opening for gas 312 and a chamber outlet opening for gas 314, wherein said chamber inlet opening for gas 312 and said chamber outlet opening for gas 314 are being in fluid connection with the interior 306 of said modular incubator chamber.

The combination of the docking station 400 with a plurality of modular incubator chambers 300 allows for incubating a viable biological material, such as an embryo or an oocyte by arranging the viable biological material in a culture dish and by accommodating the culture dish in the interior of the modular incubator chamber 300 and subsequently docking that modular incubator chamber 300 in a docking port 402 of the docking station 400.

In the present invention the term “modular incubator system” shall be construed to mean a system comprising a docking station in combination with one or more incubator chambers, wherein the one or more incubator chambers is/are configured to be docked in respective docking ports of that docking station. The modular incubator system is intended for incubation or cultivation of a viable biological material.

The incubator system comprising the docking station and one or more incubator chamber(s) in general is configured for providing some kind of interaction between the docking station and the incubator chambers being docked therein.

Such interactions may be one or more of the following: providing a gas having a desired composition to the incubator chamber(s); providing electricity to the incubator chamber(s) for powering hearing elements thereof and/or for powering a light source in the incubator chamber(s); allowing monitoring of the viable biological material being present in the incubator chamber(s), such as by means of an image capturing device which is located in the docking station.

In should be understood that within the meaning of the present application, the term “modular incubator system” shall be construed in such a way that the incubator chambers are configured to be used for incubation of a viable biological material, irrespective of whether the individual incubator chamber is being docked in a docking port of the docking station, or whether that incubator chamber is removed from the docking port of the docking station.

In this way, it is to be understood that cultivation or incubation of a viable biological material of the individual incubator chambers may take place and/or be continued even after that incubator chamber has been removed from its docking station and placed e.g. on a laboratory bench. Hereby manual manipulation operations, such as shift or control of culture or growth media, manual inspection by use of a laboratory microscope or the like can take place. Such operation are preferably carried out under a hood providing a desired gas atmosphere.

In preferred embodiment, and in order to make such manual manipulation operations practical conceivable, when the individual incubation chamber has been removed from a docking port, the incubation chamber is configured in a way that enables support on a planar, horizontal support surface. This may be attained by providing the bottom part of the incubator chamber with one or more supports or simply by making the bottom part of the incubator chamber comprise a flat surface.

In preferred embodiments the incubation chamber, in the orientation intended during use for incubation, is having its maximum dimension in a horizontal direction.

In this way the dimension of the incubation chamber in a horizontal direction is greater than the dimension in a vertical direction. Hereby, adequate stability is attended when the incubator chamber is used for incubation at a location outside a docking port of the docking station.

The individual incubator chambers may in embodiments comprise a display, such as an electronic display, for providing information relating to the identity of the viable biological material being accommodated in the incubator chamber.

It should be understood that in some embodiments the present invention does not relate to methods or uses which involve treatment of the human or animal body by surgery or diagnostic methods practiced on the human or animal body.

It should also be understood that that in other embodiments the present invention may relate to methods or uses which involve treatment of the human or animal body by surgery or diagnostic methods practiced on the human or animal body.

In one embodiment of the modular docking system according to the second aspect of the present invention and in respect of one or more of said one or more modular incubator chambers 300, and in respect of one or more of said one or more docking ports 402 of said docking station 400, the position of said chamber inlet opening for gas 312 of said modular incubator chamber 300 and the position of said docking port outlet opening for gas 404 of said docking port 402 are adapted to each other in such a way that once docking said modular incubator chamber 300 in said docking port 402, said chamber inlet opening for gas 312 of said housing 302 of said modular incubator chamber 300 and said docking port outlet opening for gas 404 of said docking port 402 will be in fluid connection; and in such a way that the position of said chamber outlet opening for gas 314 of said modular incubator chamber 300 and the position of said docking port inlet opening for gas 406 of said docking port 402 are adapted to each other in such a way that once docking said modular incubator chamber 300 in said docking port 402, said chamber outlet opening for gas 314 of said housing 302 of said modular incubator chamber 300 and said docking port inlet opening for gas 406 of said docking port 402 will be in fluid connection.

Hereby, gas having a desired composition can be delivered from the gas source 202 via the gas distribution system 204 to the interior 306 of the modular incubator chamber 300 via the docking port outlet opening for gas 404 and the chamber inlet opening for gas 312, and gas from the interior 306 of the modular incubator chamber 300 can be returned to the gas source 202 via the chamber outlet opening for gas 314 and the docking port inlet opening for gas 406.

In one embodiment of the modular docking system according to the second aspect of the present invention and in respect of one or more of said one or more modular incubator chambers 300, said chamber inlet opening for gas 312 comprises a valve 2, and said chamber outlet opening for gas 314 comprises a valve 2; wherein said valve 2 of said chamber inlet opening for gas 312 and said valve 2 of said chamber outlet opening for gas 314 each comprises a valve body 6 having a front end 10, a rear end 14 and a through-going channel 14 therein, and a spring-loaded displaceable valve element 8, wherein said displaceable valve element 8 is being arranged in said through-going channel 14; wherein said displaceable valve element 8 is being configured to be displaceable in said through-going channel 14 of said valve body 6 in such a way, that when not acted upon by an external force, said spring-loaded displaceable valve element 8 is not being displaced in said through-going channel 14 of said valve body 6, thereby making said valve attain a closed configuration blocking passage of gas through said through-going channel 14, and in such a way, that when acted upon by an external force, said spring-loaded displaceable valve element 8 is being displaced in said through-going channel 14 of said valve body 6, thereby making said valve 2 attain an open configuration, allowing passage of gas through said through-going channel 14.

Hereby can be assured that gas will only flow into the docking port 402 once a modular incubator chamber 300 is being arranged in that docking port 402. In other words, no gas will flow through the docking port 402 unless a modular incubator chamber 300 is being docked therein. Moreover, this embodiment assures that once a modular incubator chamber 300 is being removed form a docking port, no atmospheric air will enter through the chamber inlet opening for gas 312 and the chamber outlet opening for gas 314 of that chamber 300.

In one embodiment and in respect of one or more of said modular incubator chambers 300 the valve 2 is being arranged with its front end 10 pointing outward; and in respect of one or more of said docking ports 402 the valve 4 is being arranged with its front end 20 pointing outward.

In one embodiment of the modular docking system according to the second aspect of the present invention and in respect of one or more of said one or more docking ports 402 of said docking station 400, said valves 2,4 are having dimensions and geometries in such a way that once docking said modular incubator chamber in said docking port 402 of said docking station 400, said displaceable valve element 8 of said valve 2 and said displaceable valve element 18 of said valve 4 will displace each other into their respective valve bodies 6,16, thereby opening said valves 2,4 of said docking port outlet opening for gas 404 and said chamber inlet opening for gas 312; and thereby opening said valves 2,4 of said chamber outlet opening for gas 314 and said docking port inlet opening for gas 406.

Hereby, each of the two valves 2,4 will open the other valve 4,2 once being brought into contact with each other by making their front ends 10,20 meet.

In one embodiment of the modular docking system according to the second aspect of the present invention and in respect of one or more of said one or more modular incubator chambers 300, the internal parts of said modular incubator chambers 300, in terms of dimensions and geometry, are essential identical.

Hereby, the goal of having a constant pressure drop over the docking ports 402 when having a modular incubator chamber 300 docked therein, when going from one modular incubator chambers 300 to another, is further secured.

In one embodiment of the modular docking system according to the second aspect of the present invention and in respect of one or more of said one or more modular incubator chambers 300, said housing 302 of said modular incubator chamber 300 comprises a transparent window 316, and in respect of one or more docking ports 402 of said docking station 400, said docking port comprises an image capturing device 408.

Hereby capturing images of a biological material M being accommodated in the interior 306 of a modular incubator chamber 300, once being docked in said docking port 402 in enabled.

In one embodiment and in respect of one or more of said one or more modular incubator chambers 300 and in respect of one or more of said one or more docking ports 402 of said docking station 400, the position of said transparent window 316 of said modular incubator chamber 300 is adapted to the position of said image capturing device 408 in said docking port 402 in a way that enables capturing of images by said image capturing device 408 through said transparent window 316 of said modular incubator chamber 300, once said modular incubator chamber 300 is being docked in said docking port 402.

In one embodiment and in respect of one or more of said one or more modular incubator chambers 300, said transparent window 316 of said modular incubator chamber 300 is arranged at a bottom part 357 of said housing 302.

In this embodiment the image capturing device 408 will accordingly be arranged in the docking port 402 at a lower portion focusing in an upward direction.

In one embodiment of the modular docking system according to the second aspect of the present invention and in respect of one or more of said one or more modular incubator chambers 300, said transparent window 316 of said housing 302 of said modular incubator chamber is having an elongate shape, such as an elongate and linear extension extending in a direction Y transversal to said longitudinal direction X of said housing of said modular incubation chamber 300.

Hereby the image capturing device may capture images of a plurality of viable biological materials being accommodated in the same culture dish and arranged in a line having a direction Y which is transversal to said longitudinal direction X of said housing of said modular incubation chamber 300.

In one embodiment of the modular docking system according to the second aspect of the present invention and in respect of one or more of said one or more modular incubator chambers 300 and in respect of one or more of said one or more docking ports 402 of said docking station 400, said modular incubator chamber 300 is being configured to be docked in said docking port 402 with its first end 340 facing said docking port 402.

In one embodiment of the modular docking system according to the second aspect of the present invention and in respect of one or more of said one or more modular incubator chambers 300, said modular incubator chamber 300, in the interior 306 thereof, comprises a light source 372 for directing light to the area of the culture dish support 308 of said modular incubator chamber 300, thereby enabling illumination of a viable biological material in a situation of capturing images of said viable biological material.

In one embodiment said light source 372 is being attached to said lid 304 of the housing 302 of said modular incubator chamber 300, at an inner side thereof.

Hereby light can easily be directed to a viable biological material which is arranged at a lower part of the interior 306 of the modular incubator chamber 300.

In one embodiment of the modular docking system according to the second aspect of the present invention the light source 372 is being selected from the group of one or more LEDs, one or more laser diodes, one or more incandescent light bulbs.

In one embodiment of the modular docking system according to the second aspect of the present invention and in respect of one or more of said one or more modular incubator chambers 300, said culture dish support 308 is defining a planar support surface for supporting said culture dish 310.

In one embodiment of the modular docking system according to the second aspect of the present invention and in respect of one or more of said one or more modular incubator chambers 300, said housing 302 of said modular incubator chamber 300, such as at an outer portion thereof, is being provided with electric connectors 322, and wherein in respect of one or more docking ports 402 of said docking station 400, said docking port is being provided with electric connectors 410.

Hereby conveying of electric power or electric signals between said docking port 402 and said modular incubator chamber 300 is enabled.

In one embodiment of the modular docking system according to the second aspect of the present invention and in respect of one or more of said one or more modular incubator chambers 300, said lid 304 is being a hinged lid which is being connected to said housing of said modular incubator chamber via a hinge.

In one embodiment of the modular docking system according to the second aspect of the present invention in respect of one or more of said one or more modular incubator chambers 300, said housing 302 of said modular incubator chamber 300 comprises a display 324 which is being configured to display information relating to an operational status of the incubation taking place in said modular incubator chamber.

In one embodiment of the modular docking system according to the second aspect of the present invention the number of modular incubator chambers 300 of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

In one embodiment of the modular docking system according to the second aspect of the present invention and in respect of one or more of said one or more modular incubator chambers 300, said modular incubator chamber comprises an incubation chamber engagement means 326 and wherein in respect of one or more docking ports 402 of said docking station 400, said docking port comprises a docking port engagement means 414, wherein said incubation chamber engagement means 326 is being configured to enter into engagement with said docking port engagement means 414.

Hereby, easy and proper positioning and optionally also fixing said modular incubator chamber 300 in said docking port 402, as well as detaching said modular incubator chamber 300 from said docking port 402 of said docking station 400 is provided.

In one embodiment of the modular docking system according to the second aspect of the present invention and in respect of one or more of said modular incubator chambers 300, said modular incubator chamber comprises in its interior 306 an electric heating element 318 for heating the interior of said modular incubator chamber, and wherein said modular incubator chamber comprises a power source 320 for providing power to said heating element 318, wherein said electric heating element 318 is being electrically connected to said power source 320.

In an embodiment said power source 320 is being an electric power source, such as a battery, for example a rechargeable battery.

In an embodiment said heating element 318 is being thermally connected to a heat distribution element for distributing heat dissipated in said heating element; wherein said heat distribution element is being arranged, at least partly, in the interior 306 of said modular incubator chamber 300.

In an embodiment said chamber comprises a thermostat 374 and an electric thermostatic circuit 376, wherein said electric heating element 318, said power source 320 and said thermostat 374 are being electrically connected in said electric thermostatic circuit 376 so as to enable thermostatic control of the temperature inside said modular incubator chamber 300.

The above embodiments provides for upholding a desirable and predetermined and optionally also optimum temperature in the interior 306 of the modular incubator chamber 300 in a situation where the modular incubator chamber is removed from its associated docking port 402 with the view to perform visual inspection and manual replenishing, removal or exchange of growth medium to the biological materials being incubated.

In one embodiment of the modular docking system according to the second aspect of the present invention the modular incubator system 500 comprises an image processing unit 660 for image processing of images captured by said image capturing device 408, wherein said modular incubator system 500 furthermore comprises a data storage 658 for storing images captured by said image capturing units 408 and/or for storing images processed by said image processing unit 660.

An image processing unit is beneficial for manipulating the images captured, such as for adjusting contrast, for filtering and for generating time-lapse series of images.

In an embodiment of the modular incubator system according to the second aspect of the present invention said modular incubator system comprises a control unit 650 for controlling the operation thereof.

In an embodiment of the modular incubator system according to the second aspect of the present invention said control unit 650 is being coupled to an input device 652, such as an alphanumerical input device for allowing a user to provide settings input relating to a desired operational protocol of said modular incubator system.

In an embodiment of the modular incubator system according to the second aspect of the present invention said control unit 650 is being coupled to a display unit 654 for displaying, to a user, information relating to settings and/or operational status of said modular incubator system 500.

In an embodiment of the modular incubator system according to the second aspect of the present invention and in respect of one or more docking ports 402 of said docking station 400, and or in respect of a modular incubator chamber 300 being docked therein, said control unit 650 is being configured for independently controlling one or more of the following: the setting of said thermostat 374 of a modular incubator chamber 300 being docked therein, switching on and off an active light source 372 of a modular incubator chamber 300 being docked therein and/or regulating the intensity of light emitting from that active light source 372, said gas mixing control system 270; said image capturing unit 408 and/or said associated displacement device 482 of one or more of said docking ports 402 of the docking station 400 of the modular incubator system 500; said image processing unit 660.

Hereby the operation of the modular docking system 500 can easily be controlled centrally.

In an embodiment of the modular incubator system according to the second aspect of the present invention said control unit 650 is being coupled to a data processing unit 656 and optionally also to a data storage 658 for aiding in handling information during controlling of said modular incubator system.

In an embodiment of the modular incubator system according to the second aspect of the present invention said control unit 650 is being configured for conducting automatic operation of said modular incubator system 500 by independently controlling of one or more of the following: the setting of said thermostat 374 of a modular incubator chamber 300 being docked in a docking port 402, switching on and off an active light source 372 of a modular incubator chamber 300 being docked in a docking port 402 and/or regulating the intensity of light emitting from that active light source 372 of a modular incubator chamber being docked in a docking port 402, said gas mixing control system 270; said image capturing unit 408 and/or said associated displacement device 482 of one or more of said docking ports 402 of the docking station 400 of the modular incubator system 500, said gas mixing control system 270 of said docking station 400 according to predefined control instructions provided thereto, said image processing unit 660.

In an embodiment of the modular incubator system according to the second aspect of the present invention said control unit 650 is being configured for generating time lapse capturing of images by said image capturing device(s) 408.

### The Third Aspect of the Present Invention

In the third aspect, the present invention relates to a gas source 202 for providing gas to a docking station 400, wherein said gas source 202 comprises a supply gas outlet 206 to be connected to a main gas supply line 210 of a docking station 400, and wherein said gas source 202 comprises a return gas inlet 208 to be connected to a main gas return line 212 of said docking station 400; wherein said gas source 202 is as defined in respect of the first aspect of the present invention.

### The Fourth Aspect of the Present Invention

In the fourth aspect, the present invention provides a use of a docking station 400 according to the first aspect of the present invention for incubation of a viable biological material.

In one embodiment of the use according to the fourth aspect of the present invention said biological material is being an oocyte or an embryo, such as a human oocyte or a human embryo.

### The fifth aspect of the present invention

In the fifth aspect, the present invention provides a use of a modular incubator system 500 according to the second aspect of the present invention for incubation of a viable biological material.

In one embodiment of the use according to the fifth aspect of the present invention said biological material is being an oocyte or an embryo, such as a human oocyte or a human embryo.

### The Sixth Aspect of the Present Invention

In the sixth aspect, the present invention provides a method of incubating a viable biological material, wherein said method comprises:
i) providing a modular incubator system 500 according to the second aspect of the present invention;
ii) providing a viable biological material;
iii) arranging said viable biological material in a culture dish 310 and subsequently arranging said culture dish in the interior 306 of a modular incubator chamber 300 of said modular incubator system 500;
iv) docking said modular incubator chamber 300 in a docking port 402 of said docking station 400 of said modular incubator system 500;
v) allowing said viable biological material to be incubated in said modular incubator chamber 300;
vi) via said gas supply system 200 of said docking station 400, supplying gas through the interior 306 of said modular incubator chamber 300 of said modular incubator system 500.

In one embodiment of method according to the sixth aspect of the present invention said method further comprising the step of:
vi) removing said incubator chamber 300 from said docking port 402 of said docking station 400, when desired, in order to manually inspect the viable biological material, and optionally also to remove, add or exchange growth medium/media in said culture dish 310.

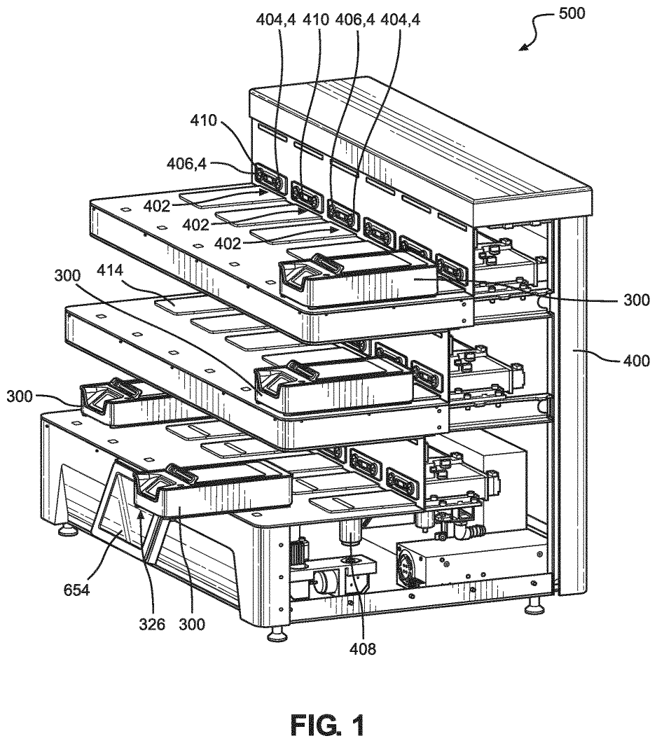

Referring now to the drawings for better illustrating the present invention in its various aspects, FIG. 1 is a perspective view illustrating the concept of a modular incubator system of the present invention comprising a docking station with a plurality of docking ports and a plurality of modular incubator chambers to be docked in the docking ports of the docking station.

FIG. 1 shows a modular incubator system 500 comprising a plurality of modular incubator chambers 300 in combination with a docking station 400.

The docking station 400 shown in FIG. 1 comprises three shelves each comprising six docking ports 402. Each docking port comprises engagement means 414 for engaging with corresponding engagement means 326 of the modular incubator chamber 300 to be docked in the docking port 402 (further described below).

FIG. 1 shows that each docking port 402 comprises a docking port outlet opening for gas 404 and a docking port inlet opening for gas 406. The openings 404 and 406 each comprises a valve 4.

Hereby is achieved that gas can be supplied from the docking port 402 to a modular incubator chamber 300 being docked into the docking port, and also that gas can be returned from the modular incubator chamber 300 into the docking port 402.

Also seen in FIG. 1 is that the docking ports 402 comprises an electric connector 410 for supplying electric power from the docking port to a modular incubator chamber 300 being docked into that docking port. Alternatively or additionally, the electric connector 410 may convey electric signals between the docking port 402 and the modular incubator chamber 300.

FIG. 1 also illustrates that below the shelf comprising the docking ports an image capturing device 408 is arranged in such a way that this image capturing device is configured for capturing images of a biological material being accommodated in a culture dish 310 which is resting on the culture dish support 308 in the interior 306 of the modular incubator chamber 300 being docked above the image capturing unit 408 as further explained below.

Hereby, morphological changes of a viable biological material can be monitored while incubating that biological material in a modular incubator chamber 300 which is being docked in a docking port 402 in a docking station 400 of the modular incubator system 500 and while a desired and predetermined composition of gas is flowing through an interior 306 of the modular incubator chamber 300.

The image capturing device comprises microscope optics for capturing close-up images.

The image capturing device(s) 408 may be configured for automatically capturing of images of the biological material being incubated in a modular incubator chamber 300.

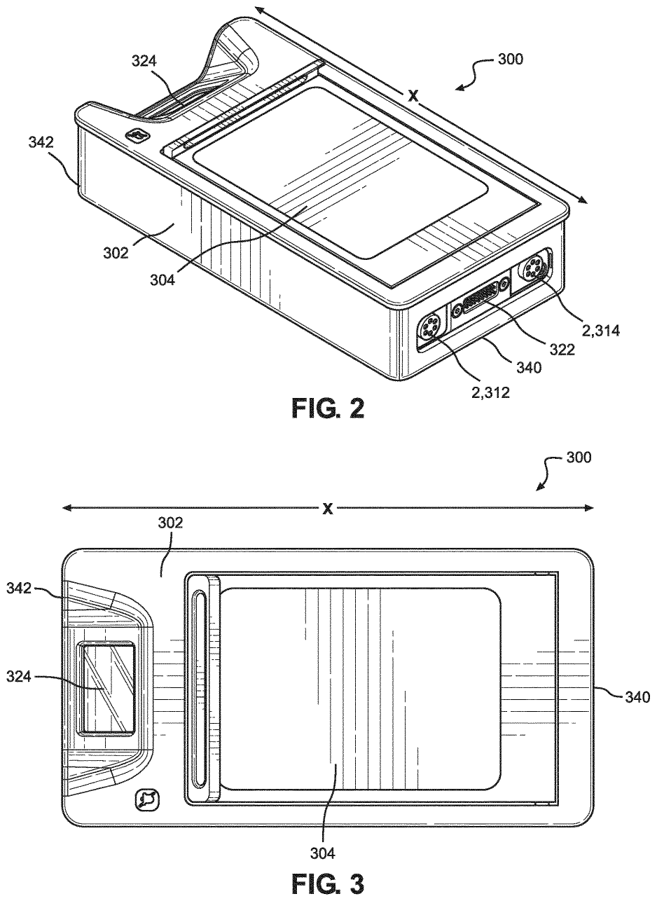

FIG. 2 is a perspective view showing a modular incubator chamber of the modular docking system of the second aspect of the present invention.

FIG. 2 shows the modular incubator chamber 300 for incubating a viable biological material. The modular incubator chamber comprises a housing 302 and the housing comprises a lid 304 which is being configured to be able to shift between an open configuration allowing access to an interior 306 of the modular incubator chamber and a closed configuration, sealing off access to the interior 306 of said modular incubator chamber.

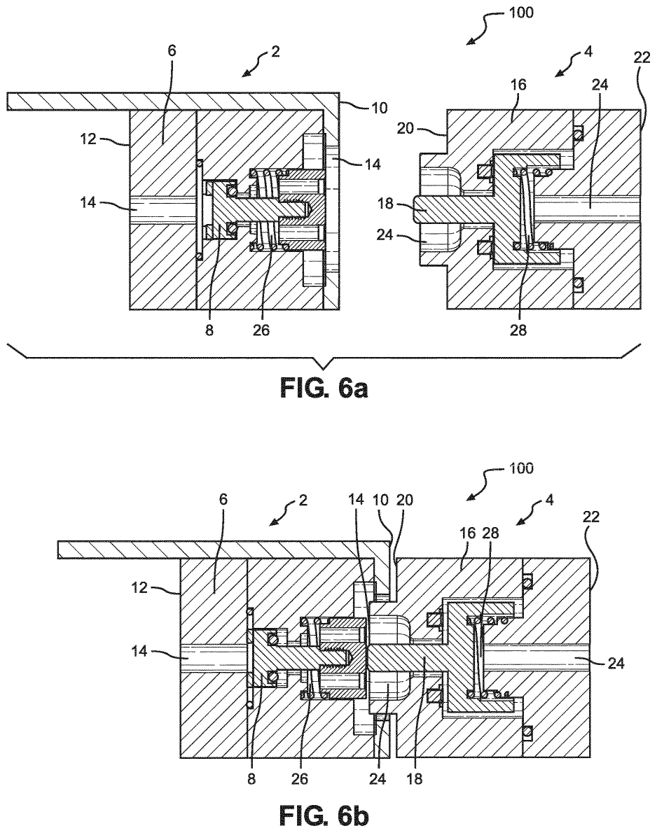

The housing 302 of the modular incubator chamber comprises a chamber inlet opening for gas 312 which is in fluid connection with the interior 306 of the modular incubator chamber, thereby allowing supplying gas into said chamber via said chamber inlet opening for gas 312. The chamber inlet opening for gas comprises a valve 2, which comprises specific features as further described below with reference to FIGS. 6a and 6b.

The housing 302 of said modular incubator chamber 300 furthermore comprises a chamber outlet opening for gas 314, which is being in fluid connection with the interior of the modular incubator chamber, thereby allowing conveying gas out of the interior 306 of modular incubator chamber 300 via the chamber outlet opening for gas 314. The chamber outlet opening for gas comprises a valve 2 which comprises specific features as further described below with reference to FIGS. 6a and 6b.

By providing the housing 302 of the modular incubator chamber 300 with a chamber inlet opening for gas 312 and an associated valve 2 and by providing the modular incubator chamber 300 with a chamber outlet opening for gas 314 and an associated valve 2 it is possible to convey a gas having a suitable and desired gas composition from a gas source 202 into the interior 306 of the modular incubator chamber 300 from a docking port 402 of the docking station 400, as will be further explained below, and further it is possible to make the gas in the interior 306 of the modular incubator chamber 300 exit the interior of the chamber through the chamber outlet opening for gas 314 and its associated valve 2 and return to the gas source 202.

Hereby a constant supply of gas having an optimum, and desired and predetermined chemical composition can be delivered to the interior of the chamber 300. This will ensure optimum incubation conditions in terms of gaseous composition of the environment in the interior 306 of the chamber 300 when incubating a biological material.

Moreover, with the incubator system according to the second aspect of the present invention it is possible to conduct a relatively large number of parallelly conducted incubations under similar conditions in the individual modular incubator chambers, while altering only one parameter from one modular incubator chamber to another. The difference in development of the viable biological material being incubated in the various modular incubator chambers can then be assigned to the one incubation parameter that is altered from one modular chamber to the other.

This allows for determining optimum incubation conditions of an embryo or an oocyte being incubated.

FIG. 2 also shows that the housing 302 of the modular incubator chamber 300 comprises a display 324 which is being configured to display information relating to details of the incubation taking place in said modular incubator chamber and that the housing 302 at a first end 340 thereof, is being provided with electric connectors 322 for providing electric power to the modular incubator chamber or for conveying electric signals between the modular incubator chamber 300 and the docking port 402 of the docking station 400.

FIG. 3 is a plan top view of the modular incubator chamber 300 illustrated in FIG. 2.

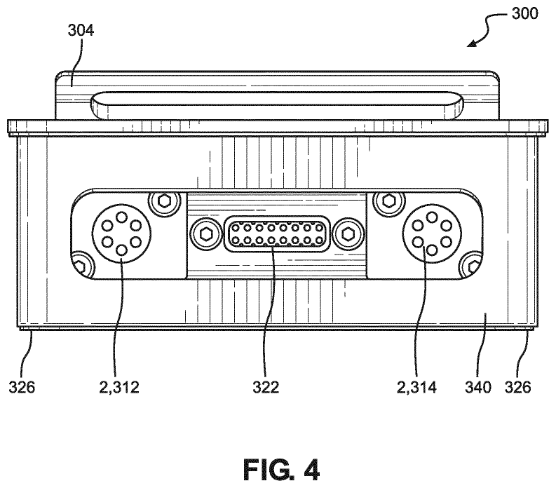

FIG. 4 is a plan rear view of the modular incubator chamber 300 illustrated in FIGS. 2 and 3 as seen from its first end.

FIG. 4 shows that the modular incubator chamber 300 comprises a chamber engagement means 326. These first engagement means 326 are configured to enter into engagement with a docking port engagement means 414 in a docking port 402 of the docking station 400.

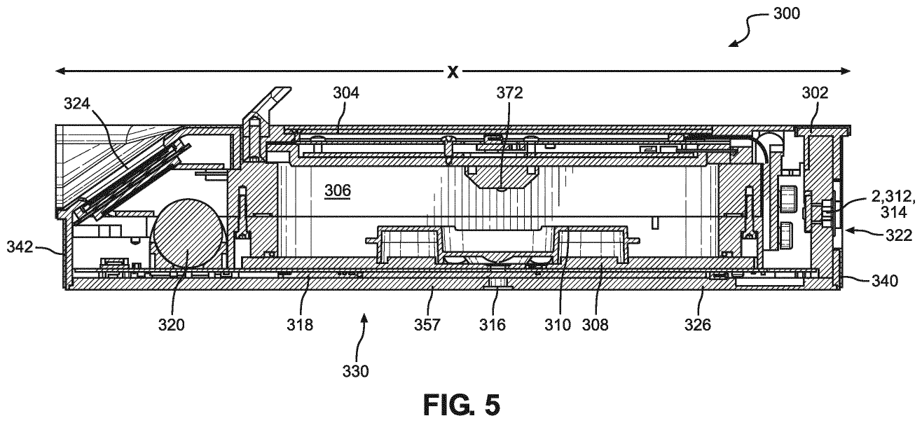

FIG. 5 is a cross-sectional view of the modular incubator chamber 300 illustrated in FIGS. 2, 3 and 4.

FIG. 5 shows that the housing 302 of the modular incubator chamber 300 comprises a transparent window 316 for allowing capturing of images of a biological material being accommodated in the interior thereof, trough said transparent window. As seen the window is arranged at the bottom 357 of the housing 302 of the modular incubator chamber 300.

The modular incubator chamber 300 also comprises, in its interior 306, an electric heating element 318 for heating the interior 306 of the modular incubator chamber. The modular incubator chamber also comprises a power source 320 in the form of a rechargeable battery for providing power to said heating element 318 which is being electrically connected to the power source 320. Thereby a desired and predetermined temperature can be upheld when the modular incubator chamber 300 is being removed from its associated docking port 402 of the docking station 400. A light source 372 is attached to an inner side of the lid 304 of the modular incubator chamber 300.

As seen in FIG. 5 the interior 306 of the modular incubator chamber 300 comprises a culture dish support 308 for positioning a culture dish 310. Hereby, one or more biological materials can be accommodated and incubated within the housing 302 of the modular incubator chamber 300.

Also seen in FIG. 5 is the chamber engagement means 326 which is being adapted to engage with docking port engagement means 414 of the docking port 402 into which the modular incubator chamber 300 is to be docked.

When such a proper positioning of the of the modular incubator chamber 300 in the docking port 402 has been attained via the chamber engagement means 326 of the chamber 300 and via the docking port engagement means 414 of the docking port 402, the relative position of the two electric connectors 410 and 322 of the docking port and the modular incubator chamber, respectively, will match pairwise so as to allow electric connection between the connectors 410 and 322. Likewise, in such situation, the two inlet openings 312 and 406 with their respective valves 2,4 and the two outlet openings 314,404 with their respective valves 2,4. will match pairwise so that passage of gas from the docking port outlet opening for gas 404 into the interior 306 of the modular incubator chamber 300 via the modular incubator chamber inlet opening for gas 312 and the valves 2,4 of the valve system 100 is enabled, and so that passage of gas from the interior 306 of the modular incubator chamber 300 is enabled via the modular incubator chamber outlet opening for gas 314 and into the docking port inlet opening for gas 406 and via the valves 2,4 arranged in these openings.

Accordingly, the modular docking system 500 of the present invention allows for continuously passing gas through the interior 306 of the modular incubator chamber from a gas source 202, when the modular incubator chamber is being docked in a docking port 402 of the docking station 400 of the modular incubator station 500.

FIGS. 6a and 6b illustrate the working modes of the valves 2,4 of the modular incubator chamber 300 and the associated docking port 402 of the docking station 400 of the docking station 500.

FIG. 6a is diagrammatic drawing illustrating a valve system 100 to be used with the modular incubator system of the present invention, where the two valves 2,4 of the valve system 100 are not engaged with each other, thereby attaining a closed configuration.

FIG. 6b is diagrammatic drawing illustrating the valve system 100 seen in FIG. 6a in which the two valves 2,4 of the valve system 100 are engaged with each other, thereby attaining an open configuration.

The valve 2 comprises a valve body 6 having a front end 10 and a rear end 12. A through-going channel 14 is arranged in the valve body 6 and a valve element 8 is being arranged in the through-going channel 14. The valve element 6 is spring-loaded by a spring 26.

The displaceable valve element 8 is being configured to be displaceable in the through-going channel 14 of the valve body 6 by the spring 26 in such a way, that when not acted upon by an external force, the spring-loaded displaceable valve element 8 is being displaced in the through-going channel 14 of said valve body 6 by the spring 26 towards the front end 10 of the valve body 6. Thereby the valve 2 attains a closed configuration blocking passage of gas through the through-going channel 14.

This situation is illustrated in FIG. 6a.

By analogy, when acted upon by an external force, the spring-loaded displaceable valve element 8 is being displaced in the through-going channel 14 of said valve body 6 towards the rear end 12 of the valve body 6, thereby making the valve 2 attain an open configuration, allowing passage of gas through said through-going channel 14.

This situation is illustrated in FIG. 6b.

As to the valve 4, FIG. 6a shows that the valve 4 comprises a valve body 16 having a front end 20 and a rear end 22. A through-going channel 24 is arranged in the valve body 16 and a valve element 18 is being arranged in the through-going channel 24. The valve element 16 is spring-loaded by a spring 28.

The displaceable valve element 18 is being configured to be displaceable in the through-going channel 24 of the valve body 16 by the spring 28 in such a way, that when not acted upon by an external force, the spring-loaded displaceable valve element 18 is being displaced in the through-going channel 24 of said valve body 16 by the spring 28 towards the first end 210 of the valve body 16. Thereby the valve 4 attains a closed configuration blocking passage of gas through the through-going channel 24. This situation is illustrated in FIG. 6a.

By analogy, when acted upon by an external force, the spring-loaded displaceable valve element 18 is being displaced in the through-going channel 24 of said valve body 16 towards the rear end 22 of the valve body 16, thereby making the valve 4 attain an open configuration, allowing passage of gas through said through-going channel 24.

This situation is illustrated in FIG. 6a.

By analogy, when acted upon by an external force, the spring-loaded displaceable valve element 18 is being displaced in the through-going channel 24 of said valve body 16 towards the rear end 22 of the valve body 16, thereby making the valve 4 attain an open configuration, allowing passage of gas through said through-going channel 24.

This situation is illustrated in FIG. 6b.

The valves 2 of the modular incubator chamber 300 and the valves 4 of the docking port 402 of the docking station are having dimensions and geometries in such a way that once docking the modular incubator chamber 300 in the docking port 402 of said docking station 400, the displaceable valve element 8 of the valve 2 and the displaceable valve element 18 of the valve 4 will displace each other into their respective valve bodies 6,16, thereby opening the valves 2,4 of said docking port outlet opening for gas 404 and said chamber inlet opening for gas 312; and also opening the valves 2,4 of the chamber outlet opening for gas 314 and the docking port inlet opening for gas 406.

Accordingly, using such valves 2,4 for the modular incubator chamber 300 and for the docking ports 402 of the docking station will automatically provide for opening the valves 2 of the modular incubator chamber 300 and the valves 4 of docking port 402, once that modular incubator chamber 300 is being docked in that docking port 402, thereby allowing passage of gas through the interior 306 of the modular incubator chamber 300, when being docking in the docking port 402 and also shutting off supply of gas into the docking port 402 and out of the modular incubator chamber 300, when that modular incubator chamber 300 is removed from the docking port.

It should be noted, that whereas the present description and the appended claims describe the modular incubator system 300 and the docking ports 402 in a way where the valves 2 are being arranged in the modular incubator system 300 and in a way where the valves 4 are being arranged in the docking ports 402, the opposite placement of the valves 2,4 is also possible.

In the above sections the general principle of a modular incubator system 500 comprising a docking station 400 with a plurality of docking ports 402 for receiving, by docking, a modular incubator chamber 300 has been described. In the sections below focus will be directed to the docking station 400 itself according to the first aspect of the present invention.

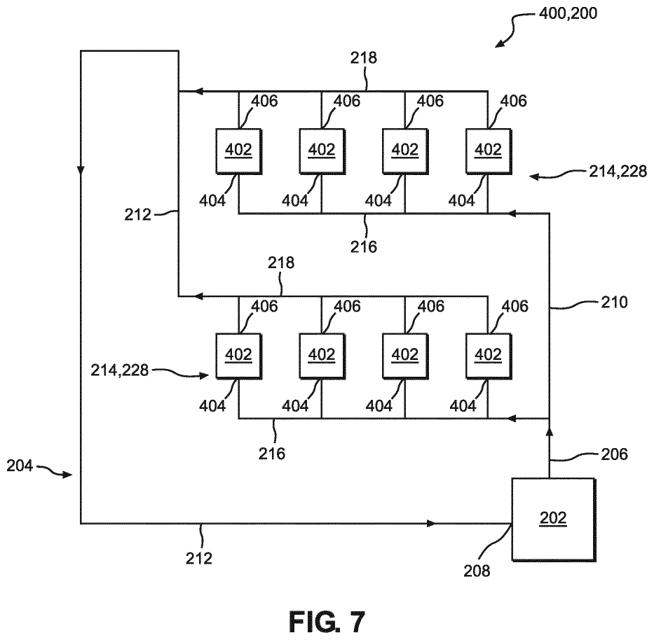

FIG. 7 is a diagram illustrating the concept of the gas supply system which in incorporated in the docking station of the modular incubator system of the present invention.

FIG. 7 shows a gas supply system 200 to be used with a modular incubator system 500 according to the present invention. The gas supply system 200 comprises a gas source 202 and a gas distribution system 204.

The gas distribution system 204 comprises a plurality of docking ports 402 each having a docking port outlet opening for gas 404 and a docking port inlet opening for gas 406.

In respect of all the docking ports, the docking port outlet openings for gas 404 are in fluid connection with an inlet manifold 216 and the docking port inlet openings for gas 406 are in fluid connection with an outlet manifold 218.

A main gas supply line 210 supplies gas from a supply gas outlet 206 of the gas source 202 to the inlet manifolds 216, and a main gas return line 212 returns gas from the outlet manifolds 218 to a return gas inlet 208 of the gas source 202.

Thereby gas can be circulated from the gas source 202 via the gas distribution system 204 to the docking ports 402 and back to the gas source 202.

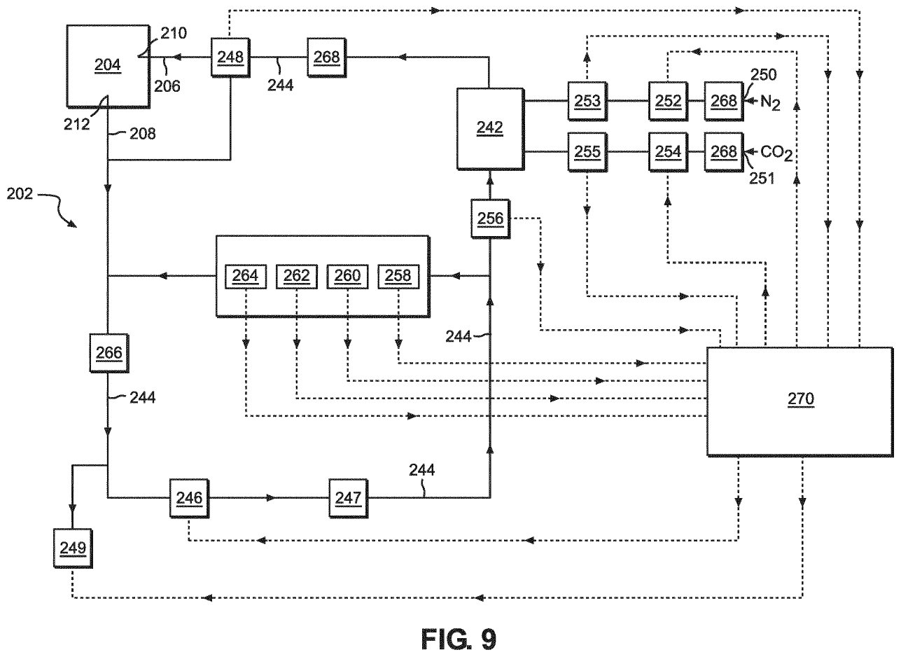

In order to secure a desired and predetermined and optimum gas composition of the gas supplied to the docking stations, the gas source is provided with specific features as disclosed with reference to FIG. 9.

However, first details of the gas distribution system 204 will be described.

As explained in the introduction to the present invention, problems may arise in maintaining a desired and optimum gas composition in the interior 306 of a modular incubator chamber 300 being docked in a docking port 400 of a docking station 400, and in particular it is difficult to maintain similar gas compositions in respect of the interior 306 of a plurality of modular incubator chambers being docked in individual docking ports.

Due to small leaks in the modular incubator chamber 300 themselves or at various points in the gas distribution system 204 it is inevitably that ambient atmospheric air will find its way to the interior 306 the modular incubator chambers 300.

Moreover, when a modular incubator chamber 300 is removed from its docking port 402 for manually performing visual inspection and manual replenishing, removal or exchange of growth medium to the biological materials being incubated therein, ambient atmospheric air will enter the interior 306 of the modular incubator chambers 300.

Finally, in case it is desired to dynamically change, over time, the gas composition to be delivered to the docking ports 402 of the docking station 400, one cannot be sure the same rate of change of gas composition will be present in respect of all modular incubator chambers 300 being docked the docking ports 402.

This may be due to the fact that the resistance encountered by the gas in its way to the docking ports and back to the gas source may vary from one docking port to another.

The present invention seeks to solve this problem.

This problem is solved by making sure that in respect of the various docking ports 402 of the docking station 400, the pressure drop from the docking port outlet opening for gas 404 to the docking port inlet opening for gas 406 is essentially the same.

This, in turn, is achieved by making sure that the travel distance for gas to and from each docking port is essentially the same.

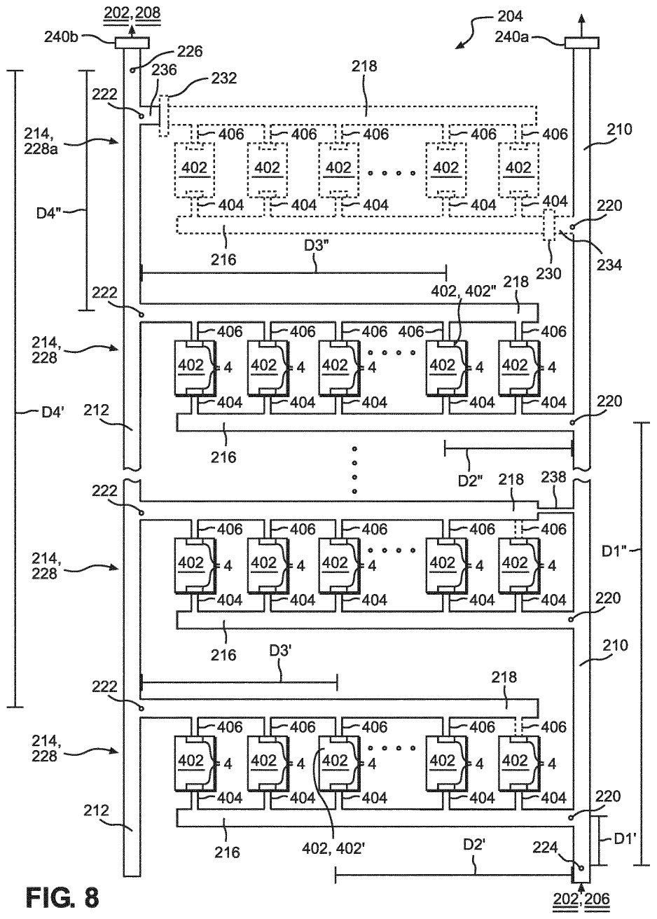

FIG. 8 illustrates an improved gas supply system for a docking station according to the first aspect of the present invention.

FIG. 8 shows part of docking station 400 for an incubator system 500. The docking station 400 comprises: a number of docking ports 402 for receiving a modular incubator chamber 300 and gas supply system 200.

The gas supply system 200 comprises a gas source 202 and a gas distribution system 204.

In respect of all docking ports of the docking station 400, the docking port 402 comprises a docking port outlet opening for gas 404 and a docking port inlet opening for gas 406.

The gas source 202 comprises a supply gas outlet 206 and a return gas inlet 208.

The gas distribution system 204 comprises a main gas supply line 210 and a main gas return line 212. The main gas supply line 210 of the gas distribution system 204 is being fluidly connected to the supply gas outlet 206 of said gas source 202 and the main gas return line 212 is being fluidly connected to the return gas inlet 208 of said gas source 202.

It is seen in FIG. 8 that the gas distribution system 204 comprises a number of manifold pairs 214. Each manifold pair comprises an inlet manifold 216 and an outlet manifold 218.

The inlet manifold 216 of each manifold pair 214 is fluidly connected to the main gas supply line 210 at an inlet manifold connection point 220

Likewise, the outlet manifold 218 of each manifold pair 214 is fluidly connected to the main gas return line 212 at an outlet manifold connection point 222.

On the main gas supply line 210 a gas supply line reference point 224 is defined which is arranged at a position upstream in relation to inlet manifold connection points 220 of all the inlet manifolds 216.

Also, on the main gas return line 212 a gas return line reference point 226 is defined which is arranged at a position downstream in relation to outlet manifold connection points 222 of all the outlet manifolds 218.

As seen in FIG. 8, each manifold pair 214 is connected to a plurality of docking ports 402 of the docking station 400 in such a way that in respect of a specific manifold pair 214, and in respect of said one or more docking ports 402 being connected thereto, the docking port outlet opening for gas 404 of said docking port 402 is being fluidly connected to the inlet manifold 216, and the docking port inlet opening for gas 406 of the docking port 402 is being fluidly connected to the outlet manifold 218.

The gas distribution system 204 is designed in such a way that in respect of all docking ports 402 of the docking station 400, the travel distance D for gas to travel from the gas supply line reference point 224 to the docking port 402 and from the docking port 402 to said gas return line reference point 226, upon conveying gas to and from the docking port 402 is being essentially equal.

The travel distance D, in respect of a specific docking port 402, is defined as:

D=D1+D2+D3+D4;
wherein D1 is defined as the distance from said supply line reference point 224 to the corresponding inlet manifold connection point 220 at said main gas supply line 210;
wherein D2 is defined as the distance from said corresponding manifold connection point 220 at said main gas supply line 210 to said docking port outlet opening for gas 404;
wherein D3 is defined as the distance from said docking port inlet opening for gas 406 to the corresponding outlet manifold connection point 222 at said main gas return line 212;
wherein D4 is defined as the distance from said outlet manifold connection point 222 at said main gas return line 212 to said return line reference point 226.

That the distance D for gas to travel between the reference points 224 and 226 is essentially equal in respect of all docking ports 402 can easily be seen by comparing the travel distance D′ for gas to travel in respect of the docking port 402′ (middle docking port in lower row) and the travel distance D″ for gas to travel in respect of the docking port 402″ (second rightmost docking port in third row from the bottom).

The travel distance D′=D1′+D2′+D3′+D4′. It is seen that this distance is equal to the travel distance D″=D1″+D2″+D3″+D4″.

The same principle applies in respect of the other docking ports 402 of the docking station 400 in FIG. 8.

FIG. 8 shows that the docking ports 402 are grouped into more groups 228 of docking ports 402 wherein, in respect of each docking port 402 belonging to a specific group 228 of docking ports 402, the docking port outlet opening for gas 404 is fluidly connected to the same inlet manifold 216; and also, in respect of each docking port 402 belonging to a specific group 228 of docking ports 402, the docking port inlet opening for gas 406 is fluidly connected to the same outlet manifold 218.

Each group of docking ports may preferably be arranged as a shelf, wherein the shelves of groups 228 of docking ports 402 are being arranged on top of each other. This arrangement of the groups of docking ports is seen in FIG. 1.

The main gas supply line 210 and said main gas return line 212 may be provided with a main line extension connector 240a,240b for enabling extension of the main gas supply line 210 and said main gas return line 212. Hereby it will be possible add an extension of the main gas supply line and an extension of the main gas return line, and thereby add one or more groups of docking ports 402, each being fluidly connected to an added inlet manifold 216 and fluidly connected to an added outlet manifold 218, wherein the added inlet manifold 216 is fluidly connected to the added extension of the main gas return line 210, and wherein the added outlet manifold 218 is fluidly connected to the added extension of said main gas return line 212 (not shown in the figures).

The extension connector 240a may arranged at the main gas supply line 210 at a position downstream in relation to all inlet manifolds, and the extension connector 240b may be arranged at the return gas supply line 212 at a position downstream in relation to all outlet manifolds as shown in FIG. 8.

Alternatively, the extension connector 240a may be arranged at the main gas supply line 210 at a position upstream in relation to all inlet manifolds, and the extension connector 240b may be arranged at the return gas supply line 212 at a position upstream in relation to all outlet manifolds.

Still alternatively, the extension connectors 240a,240b may arranged at the main gas supply line 210 and at the return gas supply line 212 at a position between the above two extreme positions.

FIG. 8 shows that the docking station 400 is being prepared for expansion by addition of one or more additional and new groups 228a of docking ports 402 (illustrated in FIG. 8 with dashed lines).

Each such new group 228a of docking ports 402 comprises a new inlet manifold 216 comprising an inlet manifold connector 230, and a new outlet manifold 218 comprising an outlet manifold connector 232.

The main gas supply line 210 of the gas distribution system 204 comprising a connector 234 which is being adapted to be connected to the inlet manifold connector 230 of the new inlet manifold 216, and the main gas return line 212 of the gas distribution system 204 comprises a connector 236 which is being adapted to be connected to the outlet manifold connector 232 of the new outlet manifold 218.

It is also seen that each new group 228a of docking ports comprises a number of new docking ports 402, wherein in respect of one or more of the new docking ports 402, the new docking port 402 comprises a docking port outlet opening for gas 404 which is being fluidly connected to the new inlet manifold 216; and wherein in respect of one or more of the new docking ports 402, the new docking port comprises a docking port inlet opening for gas 406 which is being fluidly connected to the new outlet manifold 218.

Hereby expansion of the docking station 400 with new groups 228a of docking ports 402 is possible.

Note that for the sake of simplicity the valves 4 of the new docking port outlet opening for gas 404 and of the new docking port inlet opening for gas 406 are not shown in FIG. 8.

Finally, FIG. 8 shows that the gas distribution system 204 is provided with a shunt 238 fluidly connecting the main gas supply line 210 with said main gas return line 212.

Hereby, circulation of gas in the gas distribution system in a situation where no modular incubator chamber 300 is being docked in a docking port 402 of said docking station 400 is enabled.

It is seen in FIG. 8 that the shunt 238 fluidly connects an outlet manifold 218 to the main gas supply line 210.

Generally, the shunt is preferably arranged in such a way that the travel distance for gas from the gas supply line reference point 224 to that shunt 238 plus the travel distance from that shunt 238 to the gas return line reference point 226 is essentially equal to the distance D.

In order to optimize the flow of gas in the gas distribution system 204 and the docking ports, in respect of two or more of the docking ports 402, preferably in respect of all said docking ports 402, it is preferred that the internal parts of the docking ports 402, in terms of dimensions and geometry, are essential identical.

Above, and with reference to FIGS. 8 and 9 the gas distribution system 204 of the docking station 400 has been described.

Below, the gas source 202 to be used with the docking station 400 of the first aspect of the invention will be described.

FIG. 9 is a diagram illustrating one embodiment of a design of a gas supply system comprising a gas source to be used with the docking station of the present invention.

In FIG. 9 solid lines represent flow lines for gas, whereas dashed lines represent signal lines for conveying electric signals or electric power.

FIG. 9 shows the gas distribution system 204 comprising its main gas supply line 210 and its main gas return line 212 (illustrated with the rectangle in upper left corner).

The main gas supply line 210 and the main gas return line 212 of the gas distribution system 204 is fluidly connected to a gas source 202 as described below.

The gas source 202 of said gas supply system 200 comprises a gas mixing box 242 comprising the supply gas outlet 206 and the return gas inlet 208 of the gas source.

The main gas supply line 210 of the gas distribution system 204 is being fluidly connected to the supply gas outlet 206, and the main gas return line 212 of the gas distribution system 204 is being fluidly connected to the return gas inlet 208 of the gas source 202.

Hereby a flow loop 244 comprising the gas distribution system 204 and the gas mixing box 242 is formed. The flow loop 244 comprises a pump 246 for circulating gas in that loop.

It is seen that the pump 246 is being arranged downstream in relation to the main gas return line 212. Also seen in FIG. 9 is that the flow loop 244 comprises a pump oscillation damper 247, which is being arranged immediately downstream in relation to the pump 246.

Moreover, the flow loop 244 comprises a pressure sensor in the form of a differential pressure sensor 248 for sensing the pressure of gas supplied to the main gas supply line 210, relative to the pressure in the return gas inlet line 208 of the gas distribution system 204. The pressure senor 248 is being arranged immediately upstream in relation to the main gas supply line 210 of the gas distribution system 204.

The flow loop 244 further comprises a release valve 249 for enabling pressure relief in the flow loop. The release valve is arranged immediately downstream in relation to the main gas return line 212 of the gas distribution system 402.

Also seen in FIG. 9 is that the gas mixing box 242 comprises an inlet for N₂ gas 250; and an inlet for CO₂ gas 251.

The inlet for N₂ gas 250 is fluidly connected to an N₂ valve 252 for regulating the inflow of N₂, and an N₂ mass flow sensor 253 arranged downstream of the N₂ valve 252 for sensing the amount of N₂ flowing into said gas mixing box 242.

The inlet for CO₂ gas 251 is fluidly connected to a CO₂ valve 254 for regulating the inflow of CO₂, and an CO₂ mass flow sensor 255 arranged downstream of the CO₂ valve 254 for sensing the amount of CO₂ flowing into the gas mixing box 242.

The flow loop 244 also comprises a mass flow sensor 256 arranged at an upstream position in relation to the gas mixing box 242 for sensing the amount of return gas entering the gas mixing box.

It is seen that the gas source 202 comprises an O₂ sensor 258 for sensing the concentration of O₂ exiting the gas distribution system 204; and that the gas source 202 comprises a CO2 sensor 260 for sensing the concentration of CO₂ exiting the gas distribution system 204.

The O₂ sensor and the CO₂ sensor is arranged downstream in relation to the pump 246.

A temperature sensor 262 for sensing the temperature of gas circulating in said flow loop 244 is included in the gas source 202. The temperature sensor is arranged downstream in relation to the pump 246 at a position corresponding to the position of the O₂ sensor 258.

A pressure sensor 264 for sensing the absolute pressure in the flow loop 244 is included in the gas source 202. This pressure sensor is arranged downstream in relation to the pump 246, at a position corresponding to the position of the CO₂ sensor 260.

Also seen in FIG. 9 is that the flow loop 244 comprises a UV sanitizer 266 for sanitizing gas flowing in the flow loop 244 via electromagnetic radiation in the UV range. The UV sanitizer is arranged immediately downstream in relation to the main gas return line 212.

It is seen in FIG. 9 that the gas source 202 comprises filters 268 in the form of HEPA/VOCs filters. One such a filter is arranged immediately upstream in relation to the main gas supply line 210. Another such a filter is arranged immediately upstream in relation to the inlet for N₂ gas 250 into the gas mixing box 242; and a third such a filter is being arranged immediately upstream in relation to the inlet for CO₂ gas 251 into the gas mixing box 242.

Finally, it is seen in FIG. 9 that the gas source 202 comprises a gas mixing control system 270.

It is seen that the gas mixing control system 270 is electrically connected to one or more of the following sensors for receiving sensing signals therefrom: the N₂ mass flow sensor 253 for sensing the amount of N₂ flowing into the gas mixing box; the CO₂ mass flow sensor 255 for sensing the amount of CO₂ flowing into the gas mixing box; the mass flow sensor 256 for sensing the amount of return gas entering the gas mixing box; the O₂ sensor 258 for sensing the concentration of O₂ exiting the main gas return line 212 of the gas distribution system 204; the CO₂ sensor 260 for sensing the concentration of CO₂ exiting the main gas return line 212 of the gas distribution system 204; the temperature sensor 262 for sensing the temperature circulating in the flow loop 244; the pressure sensor 264 for sensing an absolute pressure in the flow loop 244, the pressure sensor 248 for sensing the pressure of gas supplied to the gas main gas supply line 210 of the distribution system 204.

Also seen in FIG. 9 is that the gas mixing control system 270 is electrically connected to one or more of the following elements for control thereof: the N₂ valve 252 for regulating the inflow of N₂ into the gas mixing box 242; the CO₂ valve 254 for regulating the inflow of CO₂ to the gas mixing box 242; the pump 246 for circulating gas in the flow loop 244; the release valve 249.

The control of the gas source by the gas mixing control system 270 is performed in accordance with two control regimes. The first control regime relates to controlling the pressure of gas exiting the supply gas outlet 206, and the second control regime relates to controlling the concentration of CO₂ and O₂ of gas exiting the supply gas outlet 206. The two control regimes are conducted concurrently. This is further explained below.

The gas mixing control system 270 is being configured to receive input from the pressure sensor 248 and on the basis thereof control the pump 246 and optionally also activate the release valve 249 in order to maintain a desired and predetermined pressure of gas supplied to the main gas supply line 210 of the gas distribution system 204.

The gas mixing control system 270 is further configured to receive input from the mass flow sensor 256, and on the basis on this input to determine the total amount of CO₂ gas and N₂ gas needed to be supplied via the inlet for CO₂ gas 251 and via the inlet for N₂ gas 250 according to desired and predetermined criteria.

Based on the information relating to the total amount of CO₂ gas and N₂ gas needed to be supplied, as described above, the gas mixing control system 270 will be able to determine the mutual proportion of the CO₂ gas and N₂ gas.

This is performed by receiving input from the CO₂ sensor 260 and the O₂ sensor 258.

On the basis of the CO₂ concentration sensed, the gas mixing control system 270 will control the CO₂ valve 254, by transmitting a control signal thereto, and thereby regulate the inflow of CO₂ gas in order to reach a desired and predetermined CO₂ concentration.

Subsequently, the gas mixing control system 270 will on the basis of the O₂ concentration sensed, control the N₂ valve 252, by transmitting a control signal thereto, and thereby regulate the inflow of N₂ gas in order to reach a desired and predetermined O₂ concentration.

By using the gas source as disclosed above, a constant circulation of gas will be supplied to one or more modular incubator chamber 300 being docked in a respective docking port 402 of the docking station 400. By constantly regulating the inflow of CO₂ gas and N₂ gas based on sensed concentration of CO₂ and O₂ in the return gas from the gas distribution system 204, an optimum and predetermined gas composition can be maintained.

Due the design of the gas distribution system 204, an equal magnitude of flow through each modular incubator chamber 300 can be upheld, thereby minimizing variations of the composition of gas in the modular incubator chambers 300 from one chamber to another.

It should be noted that when reference is made to an upstream position relative to another position, that upstream position is construed to mean a position still within the gas source 202 and preferably not so much upstream that it passes the gas mixing box 242.

Likewise, when reference is made to a downstream position relative to another position, that downstream position is construed to mean a position still within the gas source 202 and preferably not so much downstream that it passes the gas mixing box 242.

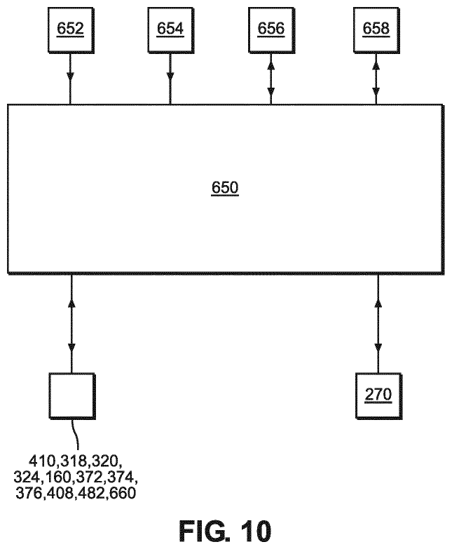

FIG. 10 is a diagram illustrating the working mode of the controlling of the modular incubator system according to the invention.

FIG. 10 shows the control unit 650 for controlling the operation of the modular incubator system 500. The control unit is coupled to an input device 652 in the form of an alphanumerical input device for allowing a user to provide settings input relating to a desired operational protocol of said modular incubator system.

A display unit 654 for displaying, to a user, information relating to settings and/or operational status of one or more of the modular incubator chambers 300 which via a docking port 402 is coupled to the control unit 650.

It is seen that the control unit 650 is coupled to a electric connectors 410 of the docking ports 402 of the docking station 400. Thereby electrical power and electric signals can be provided to one or more modular incubator chambers 300 which is/are being docked into a docking port 402 of the docking station 400 of the modular incubator system 500 via the associated connector 322 of the modular incubator chamber 300.

By being connected to the docking ports 402 of the docking station 400 it will be thereby possible, when one or more modular incubator chambers 300 is/are docked into a docking port 402 of the docking station 400, and by using the control unit 650, to independently control one or more of the following: the setting of said thermostat 374 of a modular incubator chamber 300 being docked therein, switching on and off an active light source 372 of a modular incubator chamber 300 being docked therein and/or regulating the intensity of light emitting from that active light source 372, the gas mixing control system 270; the image capturing unit 408 and/or the associated displacement device 482 of one or more of the docking ports 402 of the docking station 400 of the modular incubator system 500; and also the image processing unit 660.

The control unit 650 may comprises a CPU or other data processor 656 for processing the information involved in controlling the operation of the modular incubator system 500, e.g. by involving a computer program for handing the information involved in the controlling of the operation and the control unit 650 may also comprise a data storage 658.

Thereby automatic operation of the modular incubator system 500 may be performed in the sense that control unit 650 may inter alia independently control the temperature, the gas composition, the switching on and off of the light source 372, and the image capturing unit 408 of one or more of the modular incubator chambers.

Accordingly, with the modular incubator system 500 of the present invention, viable biological materials can be incubated in one or more modular incubators 300 which are being docked in docking ports 402 of the docking station 400, and at the same time visual monitoring of the biological material can be conducted via the image capturing device 408.

Moreover, at the same time a desired gas composition can be maintained in the interior 306 of each modular incubator chamber 300. As the modular incubator chambers comprises valves 2,4 in the respective inlet opening for gas 312 and outlet opening for gas 314, the gas atmosphere will be maintained and not disturbed by the outside atmosphere (relative to the interior 306 of the modular incubator chamber 300), even when the modular incubator chamber is removed from its respective docking port 402 of the docking station 400 of the modular incubator system 500. Upon such removal of the modular incubator chamber 300 from a docking port 402 of the docking station 400, the power source 320 and the electric heating element 318 will enable upholding the temperature in the interior 306 of the modular incubator chamber 300.

The present invention thereby allows for incubation of a biological material in the modular incubator chamber 300 and at the same time allows for visual monitoring of the morphological development of the biological material, while minimizing the detrimental effects involved in deviating from an optimized and desired gaseous atmosphere in the interior of the modular incubator chamber.

It should be noted that in respect of a number N of adjacently arranged docking ports 402 of the docking station 400, these adjacently arranged docking ports 402 may share a common image capturing device 408 in the sense that one and only one image capturing device is responsible for capturing images relating to a modular incubator chamber 300 which is being docked in one of these N adjacently arranged docking ports 402.

In such a situation, a displacement device 482 in the form of an electrically motorized suspension of the image capturing device 408 is being configured to be displaced, upon receiving a signal thereto, along a displacement track extending below these N number of adjacent docking ports 402 for enabling displacement of that common image capturing device 408 in relation to the N adjacently arranged docking ports 402 of the docking station 400. Thereby the common image capturing device 408 will be able to capture images of a biological material being accommodated in the interior 306 of a modular incubator chamber 300 being docked in any of said N docking ports 402 of the docking station 400.

Although the above embodiments have been disclosed in a way where the first valve 2 of the valve system 100 is arranged in the modular incubator chamber 300 and in such a way that the second valve 4 of the valve system 100 is arranged in the docking port 402, the opposite arrangement may also be possible. Both in respect of valves 2,4 letting gas into the chamber 300 and in respect of valves 2,4 letting gas out the chamber, or both.

It should be understood that all features and achievements discussed above and in the appended claims and clauses in relation to one aspect of the present invention and embodiments thereof apply equally well to the other aspects of the present invention and embodiments thereof.

The present invention may be defined according to one or more of the following clauses:

Clause 1. A docking station (400) for a modular incubator system (500), wherein said docking station comprises:
a number of docking ports (402) for receiving a modular incubator chamber (300); and
gas supply system (200);
wherein in respect of one or more of said docking ports (402), preferably in respect of all said docking ports of said docking station (400), said docking port comprises a docking port outlet opening for gas (404) and a docking port inlet opening for gas (406);
wherein said gas supply system (200) comprises a gas source (202) and a gas distribution system (204);
wherein said gas source (202) comprises a supply gas outlet (206) and a return gas inlet (208);
wherein said gas distribution system (204) comprises a main gas supply line (210) and a main gas return line (212);
wherein said main gas supply line (210) of said gas distribution system (204) is being fluidly connected to said supply gas outlet (206) of said gas source (202) and wherein said main gas return line (212) is being fluidly connected to said return gas inlet (208) of said gas source (202);
wherein said gas distribution system (204) comprises a number of manifold pairs (214), wherein each manifold pair comprises an inlet manifold (216) and an outlet manifold (218);
wherein said inlet manifold (216) of each manifold pair (214) is fluidly connected to said main gas supply line (210) at an inlet manifold connection point (220);
wherein said outlet manifold (218) of each manifold pair (214) is fluidly connected to said main gas return line (212) at an outlet manifold connection point (222);
wherein a gas supply line reference point (224) arranged at a position upstream in relation to inlet manifold connection points (220) of all said inlet manifolds (216) is being defined on said main gas supply line (210);
wherein a gas return line reference point (226) arranged at a position downstream in relation to outlet manifold connection points (222) of all said outlet manifolds (218) is being defined on said main gas return line (212);
wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218);
wherein said gas distribution system (204) is designed in such a way that in respect of two or more docking ports (402) of said docking station, preferably in respect of all docking ports (402) of said docking station, the travel distance D for gas to travel from said gas supply line reference point (224) to said docking port (402) and from said docking port (402) to said gas return line reference point (226) upon conveying gas to and from said docking port (402) is being essentially equal; wherein the travel distance D, in respect of a specific docking port (402), is defined as:

D=D1+D2+D3+D4;
wherein D1 is defined as the distance from said gas supply line reference point (224) to the corresponding inlet manifold connection point (220) at said main gas supply line (210);
wherein D2 is defined as the distance from said corresponding manifold connection point (220) at said main gas supply line (210) to said docking port outlet opening for gas (404);
wherein D3 is defined as the distance from said docking port inlet opening for gas (406) to the corresponding outlet manifold connection point (222) at said main gas return line (212);
wherein D4 is defined as the distance from said outlet manifold connection point (222) at said main gas return line (212) to said gas return line reference point (226).

Clause 2. A docking station (400) according to clause 1, wherein the distance D in respect of one docking port (402) is having a magnitude which is greater than the distance D in respect of another docking port (402), preferably in respect of any other docking port by 10% or less, such as 9% or less, e.g. 8% or less, for example 7% or less, such as 6% or less, e.g. 5% or less,, such as 4% or less, for example 3% or less, such as 2% or less, or 1% or less.

Clause 3. A docking station (400) according to clause 1 or 2, wherein said docking ports (402) are grouped into one or more groups (228) of docking ports (402) wherein, in respect of each docking port (402) belonging to a specific group (228) of docking ports (402), said docking port outlet opening for gas (404) is fluidly connected to the same inlet manifold (216); and wherein in respect of each docking port (402) belonging to a specific group (228) of docking ports (402), said docking port inlet opening for gas (406) is fluidly connected to the same outlet manifold (218).

Clause 4. A docking station (400) according to clause 3, wherein the number of groups (228) of docking ports (402) is selected from the ranges 1-20 or more, such as 2-19, for example 3-18, such as 4-17, such as 5-16, e.g. 6-15, such as 7-14, e.g. 8-13, such as 9-12 or 10-11.

Clause 5. A docking station (400) according to clause 3 or 4, wherein the number of docking ports 402 in each group (228) of docking ports (402) independently is being selected from the ranges 2-25 or more, such as 3-24, for example 4-23, e.g. 5-22, such as 6-21, e.g. 7-20, for example 8-19, such as 9-18, for example 10-17, such as 11-16, e.g. 12-15 or 13-14.

Clause 6. A docking station (400) according to any of the clauses 3-5, wherein the number of groups (228) of docking ports (402) is two or more, and wherein each group of docking ports is arranged as a shelf, wherein said shelves of groups (228) of docking ports (402) are being arranged on top of each other.

Clause 7. A docking station (400) according to any of the preceding clauses, wherein said main gas supply line (210) is provided with a main line extension connector (240a) for enabling extension of said main gas supply line (210), and wherein said main gas return line (212) is provided with a main line extension connector (240b) for enabling extension of said main gas return line (212) with the view to add an extension of the main gas supply line and an extension of the main gas return line, and thereby enable adding one or more groups (228) of docking ports (402), each being fluidly connected to an added inlet manifold (216) and fluidly connected to an added outlet manifold (218), wherein said added inlet manifold (216) is fluidly connected to said added extension of said main gas return line (210), and wherein said added outlet manifold (218) is fluidly connected to said added extension of said main gas return line (212).

Clause 8. A docking station (400) according to clause 7, wherein said extension connector (240a) is arranged at said main gas supply line (210) at a position downstream in relation to all inlet manifolds, and wherein said extension connector (240b) is arranged at said return gas supply line (212) at a position downstream in relation to all outlet manifolds; or wherein said extension connector (240a) is arranged at said main gas supply line (210) at a position upstream in relation to all inlet manifolds, and wherein said extension connector (240b) is arranged at said return gas supply line (212) at a position upstream in relation to all outlet manifolds; or wherein said extension connectors (240a,240b) are arranged at said main gas supply line (210) and said return gas supply line (212) at a position therebetween.

Clause 9. A docking station (400) according to any of the clauses 3-8, wherein said docking station is being prepared for expansion by addition of one or more additional and new groups (228a) of docking ports (402), wherein each such new group (228a) of docking ports (402) comprises a new inlet manifold (216) comprising an inlet manifold connector (230), and a new outlet manifold (218) comprising an outlet manifold connector (232); wherein said main gas supply line (210) of said gas distribution system (204) comprising one or more connectors (234) which is/are being adapted to be connected to said inlet manifold connector (230) of said new inlet manifold (216); and wherein said main gas return line (212) of said gas distribution system (204) comprises one or more connectors (236) which is/are being adapted to be connected to said outlet manifold connector (232) of said new outlet manifold (218); wherein each new group (228a) of docking ports comprises a number of new docking ports (402), wherein in respect of one or more of said new docking ports (402), said new docking port (402) comprises a docking port outlet opening for gas (404) which is being fluidly connected to said new inlet manifold (216); and wherein in respect of one or more of said new docking ports (402), said new docking port comprises a docking port inlet opening for gas (406) which is being fluidly connected to said new outlet manifold (218); thereby enabling expansion of said docking station (400) with new groups (228a) of docking ports (402).

Clause 10. A docking station (400) according to any of the preceding clauses, wherein in respect of one or more of said docking ports (402), preferably in respect of all said docking ports, said docking port outlet opening for gas (404) comprises a valve (4), and said docking port inlet opening for gas (406) comprises a valve (4); wherein said valve (4) of said docking port outlet opening for gas (404) and said valve (4) of said docking port inlet opening for gas (406) each comprises a valve body (16) having a front end (20), a rear end (22) and a through-going channel (24) therein, and a spring-loaded displaceable valve element (18), wherein said displaceable valve element (18) is being arranged in said through-going channel (24); wherein said displaceable valve element (18) is being configured to be displaceable in said through-going channel (24) of said valve body (16) in such a way, that when not acted upon by an external force, said spring-loaded displaceable valve element (18) is not being displaced in said through-going channel (24) of said valve body (16), thereby making said valve attain a closed configuration blocking passage of gas through said through-going channel (24), and in such a way, that when acted upon by an external force, said spring-loaded displaceable valve element (18) is being displaced in said through-going channel (24) of said valve body (16), thereby making said valve (4) attain an open configuration, allowing passage of gas through said through-going channel (24).

Clause 11. A docking station (400) according to any of the preceding clauses, wherein said gas distribution system (204) comprises one or more shunts (238) fluidly connecting said main gas supply line (210) with said main gas return line (212), thereby enabling circulation of gas in said gas distribution system in a situation where no modular incubator chamber (300) is being docked in a docking port (402) of said docking station (400).

Clause 12. A docking station (400) according to clause 11, wherein one or more of said shunts (238) fluidly connects an outlet manifold (218) to said main gas supply line (210); or wherein one or more of said shunts (238) fluidly connects an inlet manifold (216) to said main gas return line (212); or wherein one or more of said shunts (238) fluidly connects an inlet manifold (216) to an outlet manifold (218).

Clause 13. A docking station (400) according to clause 11 or 12, wherein in respect of one or more of said shunts, said shunt is arranged in such a way that the travel distance for gas from said gas supply line reference point (224) to said shunt plus the travel distance from said shunt to said gas return line reference point (226) is essentially equal to the distance D.

Clause 14. A docking station (400) according to any of the preceding clauses, wherein in respect of two or more of said docking ports (402), preferably in respect of all said docking ports (402), the internal parts of said docking ports (402), in terms of dimensions and geometry, are essential identical.

Clause 15. A docking station (400) according to any of the preceding clauses, wherein in respect of two or more of said docking ports (402), preferably in respect of all said docking ports (402), said docking port outlet opening for gas (404) comprises a flow restrictor for restricting the magnitude of flow of gas flowing into said docking port (402).

Clause 16. A docking station (400) according to clause 15, wherein said flow restrictor comprises a tube through which the gas is conveyed to said docking port (402), wherein said tube optionally is having a cross-sectional area selected from the ranges of 0.2-8 mm², such as 0.5-7 mm², for example 1-6 mm², such as 2-5 mm² or 3-4 mm²; and/or wherein the length of said tube optionally is selected from the ranges of 5-30 mm, such as 8-25 mm, for example 10-22 mm, e.g. 15-20 mm.

Clause 17. A docking station (400) according to any of the preceding clauses, wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

Clause 18. A docking station (400) according to clause 17, wherein in respect of one or more specific docking ports (402) of said docking station (400), said specific docking port comprises its own dedicated image capturing device (408) which is configured to only capture images relating to a modular incubator chamber (300) which is being docked in said specific docking port (402).

Clause 19. A docking station (400) according to clause 17 or 18, wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482), such as an electrically driven and remotely controlled displacement device (482) for enabling displacement of said common image capturing device (408) in relation to said N adjacently arranged docking ports (402) of said docking station (400).

Clause 20. A docking station (400) according to clause 19, wherein said number N is being an integer selected in the ranges of 2-25 or more, such as 4-22, for example 6-20, such as 8-18, such as 10-16 or 12-14.

Clause 21. A docking station (400) according to any of the clauses 17-20, wherein one or more of said image capturing devices (408) of said docking ports (402) comprise(s) microscopic optics so as to enable capturing of microscope images.

Clause 22. A docking station (400) according to any of the preceding clauses, wherein said gas source (202) of said gas supply system (200) comprises a gas mixing box (242) comprising said supply gas outlet (206) and said return gas inlet (208) of said gas source, wherein said main gas supply line (210) of said gas distribution system (204) is being fluidly connected to said supply gas outlet (206), and wherein said main gas return line (212) of said gas distribution system (204) is being fluidly connected to said return gas inlet (208) of said gas source (202), thereby forming a flow loop (244) comprising said gas distribution system (204) and said gas mixing box (242); wherein said flow loop comprises a pump (246) for circulating gas in said loop.

Clause 23. A docking station (400) according to clause 22, wherein said pump (246) is being arranged downstream in relation to said main gas return line (212).

Clause 24. A docking station (400) according to clause 22 or 23, wherein said flow loop (244) comprises a pump oscillation damper (247), wherein said pump oscillation damper optionally is being arranged immediately downstream in relation to said pump (246).

Clause 25. A docking station (400) according to any of the clauses 22-24, wherein said flow loop (244) comprises a pressure sensor, such as a differential pressure sensor (248) for sensing the pressure of gas supplied to said main gas supply line (210) of said gas distribution system (204), wherein said pressure senor (248) optionally is being arranged immediately upstream in relation to said main gas supply line (210) of said gas distribution system (204).

Clause 26. A docking station (400) according to clause 25, wherein said pressure sensor (248) is being a differential pressure sensor, sensing a pressure relative to the pressure of the return gas inlet (208).

Clause 27. A docking station (400) according to any of the clauses 22-26, wherein said flow loop (244) comprises a release valve (249) for enabling pressure relief in said flow loop, wherein said release valve optionally is being arranged immediately downstream in relation to said main gas return line (212) of said gas distribution system (402).

Clause 28. A docking station (400) according to any of the clauses 22-27, wherein said gas mixing box (242) comprises an inlet for N₂ gas (250); and an inlet for CO₂ gas (251), wherein said inlet for N₂ gas (250) is fluidly connected to an N₂ valve (252) for regulating the inflow of N₂, and an N₂ mass flow sensor (253) arranged downstream of said N₂ valve (252) for sensing the amount of N₂ flowing into said gas mixing box (242); and wherein said inlet for CO₂ gas (251) is fluidly connected to a CO₂ valve (254) for regulating the inflow of CO₂, and an CO₂ mass flow sensor (255) arranged downstream of said CO₂ valve (254) for sensing the amount of CO₂ flowing into said gas mixing box (242).

Clause 29. A docking station (400) according to any of the clauses 22-28 wherein said flow loop (244) comprises a mass flow sensor (256) arranged at an upstream position in relation to said gas mixing box (242) for sensing the amount of return gas entering said gas mixing box.

Clause 30. A docking station (400) according to any of the clauses 22-29, wherein said gas source (202) comprises an O₂ sensor 258 for sensing the concentration of O₂ exiting said gas distribution system (204); and wherein said gas source (202) comprises a CO₂ sensor (260) for sensing the concentration of CO₂ exiting said gas distribution system (204), wherein said O₂ sensor and/or said CO₂ sensor optionally is/are being arranged downstream in relation to said pump (246).

Clause 31. A docking station (400) according to any of the clauses 22-30, wherein said gas source (202) comprises a temperature sensor (262) for sensing the temperature of gas circulating in said flow loop (244), wherein said temperature sensor optionally is being arranged downstream in relation to said pump (246), preferably at a position corresponding to the position of said O₂ sensor (258).

Clause 32. A docking station (400) according to any of the clauses 22-31, wherein said gas source (202) comprises a pressure sensor (264) for sensing the absolute pressure in said flow loop (244) wherein said pressure sensor optionally is being arranged downstream in relation to said pump (246), preferably at a position corresponding to the position of said CO₂ sensor (260).

Clause 33. A docking station (400) according to any of the clauses 22-32, wherein said flow loop (244) comprises a UV sanitizer (266) for sanitizing gas flowing in said flow loop (244) via electromagnetic radiation in the UV range, wherein said UV sanitizer optionally being arranged immediately downstream in relation to said main gas return line (212).

Clause 34. A docking station (400) according to any of the clauses 22-33, wherein said gas source (202) comprises one or more filters (268), such as HEPA and/or VOCs filters, wherein such a filter is being arranged immediately upstream in relation to said main gas supply line (210), and/or wherein such a filter is being arranged immediately upstream in relation to the inlet for N₂ gas (250) into said gas mixing box (242); and/or wherein such a filter is being arranged immediately upstream in relation to the inlet for CO₂ gas (251) into said gas mixing box (242).

Clause 35. A docking station (400) according to any of the clauses 22-33, wherein said gas source (202) comprises a gas mixing control system (270), wherein said gas mixing control system is electrically connected to one or more of the following sensors for receiving sensing signals therefrom: said N₂ mass flow sensor (253) for sensing the amount of N₂ flowing into said gas mixing box; said CO₂ mass flow sensor (255) for sensing the amount of CO₂ flowing into said gas mixing box; said mass flow sensor (256) for sensing the amount of return gas entering said gas mixing box; said O₂ sensor (258) for sensing the concentration of O₂ exiting said main gas return line (212) of said gas distribution system (204); said CO₂ sensor (260) for sensing the concentration of CO₂ exiting said main gas return line (212) of said gas distribution system (204); said temperature sensor (262) for sensing the temperature circulating in said flow loop (244); said pressure sensor (264) for sensing an absolute pressure in said flow loop (244), said pressure sensor (248) for sensing the pressure of gas supplied to said gas main gas supply line (210) of said distribution system (204).

Clause 36. A docking station (400) according to clauses 35, wherein said gas mixing control system (270) is electrically connected to one or more of the following elements for control thereof: said N₂ valve (252) for regulating the inflow of N₂ into said gas mixing box (242); said CO₂ valve (254) for regulating the inflow of CO₂ to said gas mixing box (242); said pump (246) for circulating gas in said flow loop (244); said release valve (249).

Clause 37. A docking station (400) according to clause 35 or 36, wherein said gas mixing control system (270) is being configured to receive input from said pressure sensor (248) and on the basis thereof control said pump (246), optionally also to activate said release valve (249) in order to maintain a desired and predetermined pressure of gas supplied to said main gas supply line (210) of said gas distribution system (204).

Clause 38. A docking station (400) according to any of the clauses 35-37, wherein said gas mixing control system (270) is being configured to receive input from said mass flow sensor (256), and on the basis on said input to determine the total amount of CO₂ gas and N₂ gas needed to be supplied via said inlet for CO₂ gas (251) and via said inlet for N₂ gas (250) according to desired and predetermined criteria.

39. A docking station (400) according to any of the clauses 35-38, wherein said gas mixing control system (270) is being configured to receive input from said CO₂ sensor (260) and said O₂ sensor (258), and on the basis of the CO₂ concentration sensed, is configured to control said CO₂ valve (254), by transmitting a control signal thereto, and thereby regulating the inflow of CO₂ gas in order to reach a desired and predetermined CO₂ concentration, and wherein subsequently, said gas mixing control system (270) on the basis of the O₂ concentration sensed, is configured to control said N₂ valve (252), by transmitting a control signal thereto, and thereby regulating the inflow of N₂ gas in order to reach a desired and predetermined O₂ concentration.

Clause 40. A docking station (400) according to any of the clauses 35-39, wherein said gas mixing control system (270) is configured to use the input from said temperature sensor (262) for compensating the temperature sensitivity of said O₂ sensor (258).

Clause 41. A docking station (400) according to any of the clauses 35-40, wherein said gas mixing control system (270) is configured to use the input from said pressure sensor (264) for compensating the pressure sensitivity of said CO₂ sensor (260).

Clause 42. A docking station (400) according to any of the clauses 35-41, wherein said gas mixing control system (270) is being configured to maintain a pressure of gas supplied to said main gas supply line (210) of said gas distribution system (204), relative to the ambient atmospheric pressure, of 3-20 mbar, such as 5-18 mbar, such as 10-15 mbar above that ambient atmospheric pressure.

Clause 43. A docking station (400) according to any of the clauses 35-42, wherein said gas mixing control system (270) is being configured to maintain a CO₂ concentration of gas entering said main gas supply line (210) of said gas distribution system (204) in the range of 5-10%, such as 6-9% or 7-8%; and/or an O₂ concentration of gas entering said main gas supply line (210) of said gas distribution system 204 in the range of 5-10%, such as 6-9% or 7-8%.

Clause 44. A modular incubator system (500) comprising a docking station (400) according to any of the clauses 1-43 in combination with one or more modular incubator chambers (300); wherein in respect of one or more of said one or more modular incubator chambers (300), said modular incubator chamber (300) comprises:
a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said modular incubator chamber (300) comprises a chamber inlet opening for gas (312) and a chamber outlet opening for gas (314), wherein said chamber inlet opening for gas (312) and said chamber outlet opening for gas (314) are being in fluid connection with the interior (306) of said modular incubator chamber.

Clause 45. A modular incubator system (500) according to clause 44, wherein in respect of one or more of said one or more modular incubator chambers (300), and in respect of one or more of said one or more docking ports (402) of said docking station (400), the position of said chamber inlet opening for gas (312) of said modular incubator chamber (300) and the position of said docking port outlet opening for gas (404) of said docking port (402) are adapted to each other in such a way that once docking said modular incubator chamber (300) in said docking port (402), said chamber inlet opening for gas (312) of said housing (302) of said modular incubator chamber (300) and said docking port outlet opening for gas (404) of said docking port (402) will be in fluid connection; and in such a way that the position of said chamber outlet opening for gas (314) of said modular incubator chamber (300) and the position of said docking port inlet opening for gas (406) of said docking port (402) are adapted to each other in such a way that once docking said modular incubator chamber (300) in said docking port (402), said chamber outlet opening for gas (314) of said housing (302) of said modular incubator chamber (300) and said docking port inlet opening for gas (406) of said docking port (402) will be in fluid connection.

Clause 46. A modular incubator system (500) according to clause 44 or 45, wherein in respect of one or more of said one or more modular incubator chambers (300), said chamber inlet opening for gas (312) comprises a valve (2), and said chamber outlet opening for gas (314) comprises a valve (2); wherein said valve (2) of said chamber inlet opening for gas (312) and said valve (2) of said chamber outlet opening for gas (314) each comprises a valve body (6) having a front end (10), a rear end (12) and a through-going channel 14 therein, and a spring-loaded displaceable valve element (8), wherein said displaceable valve element (8) is being arranged in said through-going channel (14); wherein said displaceable valve element (8) is being configured to be displaceable in said through-going channel (14) of said valve body (6) in such a way, that when not acted upon by an external force, said spring-loaded displaceable valve element (8) is not being displaced in said through-going channel (14) of said valve body (6), thereby making said valve attain a closed configuration blocking passage of gas through said through-going channel (14), and in such a way, that when acted upon by an external force, said spring-loaded displaceable valve element (8) is being displaced in said through-going channel (14) of said valve body (6), thereby making said valve (2) attain an open configuration, allowing passage of gas through said through-going channel (14).

Clause 47. A modular incubator system (500) according to clause 46, wherein in respect of one or more of said modular incubator chambers (300) said valve (2) is being arranged with its front end (10) pointing outward; and wherein in respect of one or more of said docking ports (402) said valve (4) is being arranged with its front end (20) pointing outward.

Clause 48. A modular incubator system (500) according to clause 46 or 47, wherein in respect of one or more of said one or more docking ports (402) of said docking station (400), said docking port(s) is/are as defined in clause 10, and wherein in respect of one or more of said one or more modular incubator chambers (300) said modular incubator chamber is as defined in clause 46, wherein said valves (2,4) are having dimensions and geometries in such a way that once docking said modular incubator chamber in said docking port (402) of said docking station (400), said displaceable valve element (8) of said valve (2) and said displaceable valve element (18) of said valve (4) will displace each other into their respective valve bodies (6,16), thereby opening said valves (2,4) of said docking port outlet opening for gas (404) and said chamber inlet opening for gas (312); and thereby opening said valves (2,4) of said chamber outlet opening for gas (314) and said docking port inlet opening for gas (406).

Clause 49. A modular incubator system (500) according to any of the clauses 44-48, wherein in respect of one or more of said one or more modular incubator chambers (300), the internal parts of said modular incubator chambers (300), in terms of dimensions and geometry, are essential identical.

Clause 50. A modular incubator system (500) according to any of the clauses 44-49, wherein in respect of one or more of said one or more modular incubator chambers (300), said housing (302) of said modular incubator chamber (300) comprises a transparent window (316), and wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

Clause 51. A modular incubator system (500) according to clause 50, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

Clause 52. A modular incubator system (500) according to clause 50 or 51, wherein in respect of one or more of said one or more modular incubator chambers (300), said transparent window (316) of said modular incubator chamber (300) is arranged at a bottom part (357) of said housing (302).

Clause 53. A modular incubator system (500) according to any of the clauses 50-52, wherein in respect of one or more of said one or more modular incubator chambers (300), said transparent window (316) of said housing (302) of said modular incubator chamber is having an elongate shape, such as an elongate and linear extension extending in a direction Y transversal to said longitudinal direction X of said housing of said modular incubation chamber (300).

Clause 54. A modular incubator system (500) according to any of the clauses 44-53, wherein in respect of one or more of said one or more modular incubator chambers (300) and in respect of one or more of said one or more docking ports (402) of said docking station (400), said modular incubator chamber (300) is being configured to be docked in said docking port (402) with its first end (340) facing said docking port (402).

Clause 55. A modular incubator system (500) according to any of the clauses 44-54, wherein in respect of one or more of said one or more modular incubator chambers (300), said modular incubator chamber (300), in the interior (306) thereof, comprises a light source (372) for directing light to the area of the culture dish support (308) of said modular incubator chamber (300), thereby enabling illumination of a viable biological material in a situation of capturing images of said viable biological material.

Clause 56. A modular incubator system (500) according to clause 55, wherein said light source (372) is being attached to said lid (304) of the housing (302) of said modular incubator chamber (300), at an inner side thereof.

Clause 57. A modular incubator system (500) according to clause 55 or 56, wherein said light source (372) is being selected from the group of one or more LEDs, one or more laser diodes, one or more incandescent light bulbs.

Clause 58. A modular incubator system (500) according to any of the clauses 44-57, wherein in respect of one or more of said one or more modular incubator chambers (300), said culture dish support (308) is defining a planar support surface for supporting said culture dish (310).

Clause 59. A modular incubator system (500) according to any of the clauses 44-58, wherein in respect of one or more of said one or more modular incubator chambers (300), said housing (302) of said modular incubator chamber (300), such as at an outer portion thereof, is being provided with electric connectors (322), and wherein in respect of one or more docking ports (402) of said docking station (400), said docking port is being provided with electric connectors (410), thereby enabling conveying of electric power or electric signals between said docking port (402) and said modular incubator chamber (300).

Clause 60. A modular incubator system (500) according to any of the clauses 44-59, wherein in respect of one or more of said one or more modular incubator chambers (300), said lid (304) is being a hinged lid which is being connected to said housing of said modular incubator chamber via a hinge.

Clause 61. A modular incubator system (500) according to any of the clauses 44-60, wherein in respect of one or more of said one or more modular incubator chambers (300), said housing (302) of said modular incubator chamber (300) comprises a display (324) which is being configured to display information relating to an operational status of the incubation taking place in said modular incubator chamber.

Clause 62. A modular incubator system (500) according to any of the clauses 44-61, wherein the number of modular incubator chambers (300) of said modular incubator system (500) is selected from the ranges 1-100, such as 2-95, for example 5-90, e.g. 10-85, such as 15-80, for example 20-75, e.g. 25-70, 30-65, such as 35-60, e.g. 40-55 or 45-50.

Clause 63. A modular incubator system (500) according to any of the clauses 44-62, wherein in respect of one or more of said one or more modular incubator chambers (300), said modular incubator chamber comprises an incubation chamber engagement means (326) and wherein in respect of one or more docking ports (402) of said docking station (400), said docking port comprises a docking port engagement means (414), wherein said incubation chamber engagement means (326) is being configured to enter into engagement with said docking port engagement means (414) so as to provide easy and proper positioning and optionally also fixing said modular incubator chamber (300) in said docking port (402), as well as detaching said modular incubator chamber (300) from said docking port (402) of said docking station (400).

Clause 64. A modular incubator system (500) according to any of the clauses 44-63, wherein in respect of one or more of said modular incubator chambers (300), said modular incubator chamber comprises in its interior (306) an electric heating element (318) for heating the interior of said modular incubator chamber, and wherein said modular incubator chamber comprises a power source (320) for providing power to said heating element (318), wherein said electric heating element (318) is being electrically connected to said power source (320).

Clause 65. A modular incubator system (500) according to clause 64, wherein said power source (320) is being an electric power source, such as a battery, for example a rechargeable battery.

Clause 66. A modular incubator system (500) according to any of the clauses 65 or 65, wherein said heating element (318) is being thermally connected to a heat distribution element for distributing heat dissipated in said heating element; wherein said heat distribution element is being arranged, at least partly, in the interior (306) of said modular incubator chamber (300).

Clause 67. A modular incubator system (500) according to any of the clauses 64-66, wherein said chamber comprises a thermostat (374) and an electric thermostatic circuit (376), wherein said electric heating element (318), said power source (320) and said thermostat (374) are being electrically connected in said electric thermostatic circuit (376) so as to enable thermostatic control of the temperature inside said modular incubator chamber (300).

Clause 68. A modular incubator system (500) according to any of the clauses 44-67, wherein said modular incubator system (500) comprises an image processing unit (660) for image processing of images captured by said image capturing device (408), wherein said modular incubator system (500) furthermore comprises a data storage (658) for storing images captured by said image capturing units 408 and/or for storing images processed by said image processing unit (660).

Clause 69. A modular incubator system 500 according to any of the clauses 44-68, wherein said modular incubator system comprises a control unit 650 for controlling the operation thereof.

Clause 70. A modular incubator system (500) according to clause 69, wherein said control unit (650) is being coupled to an input device (652), such as an alphanumerical input device for allowing a user to provide settings input relating to a desired operational protocol of said modular incubator system.

Clause 71. A modular incubator system (500) according to clause 69 or 70, wherein said control unit (650) is being coupled to a display unit (654) for displaying, to a user, information relating to settings and/or operational status of said modular incubator system (500).

Clause 72. A modular incubator system (500) according to any of the clauses 69-71, wherein in respect of one or more docking ports (402) of said docking station (400), and or in respect of a modular incubator chamber (300) being docked therein, said control unit (650) is being configured for independently controlling one or more of the following: the setting of said thermostat (374) of a modular incubator chamber (300) being docked therein, switching on and off an active light source (372) of a modular incubator chamber (300) being docked therein and/or regulating the intensity of light emitting from that active light source (372), said gas mixing control system (270); said image capturing unit (408) and/or said associated displacement device (482) of one or more of said docking ports (402) of the docking station (400) of the modular incubator system (500); said image processing unit (660).

Clause 73. A modular incubator system (500) according to any of the clauses 69-72, wherein said control unit (650) is being coupled to a data processing unit (656) and optionally also to a data storage (658) for aiding in handling information during controlling of said modular incubator system.

Clause 74. A modular incubator system (500) according to any of the clauses 69-73, wherein said control unit (650) is being configured for conducting automatic operation of said modular incubator system (500) by independently controlling of one or more of the following: the setting of said thermostat (374) of a modular incubator chamber (300) being docked in a docking port (402), switching on and off an active light source (372) of a modular incubator chamber (300) being docked in a docking port (402) and/or regulating the intensity of light emitting from that active light source (372) of a modular incubator chamber being docked in a docking port (402), said gas mixing control system (270); said image capturing unit (408) and/or said associated displacement device (482) of one or more of said docking ports (402) of the docking station (400) of the modular incubator system (500), said gas mixing control system (270) of said docking station (400) according to predefined control instructions provided thereto, said image processing unit (660).

Clause 75. A modular incubator system (500) according to any of the clauses 69-74, wherein said control unit (650) is being configured for effecting time lapse capturing of images by said image capturing device(s) (408).

Clause 76. A gas source (202) for providing gas to a docking station (400), wherein said gas source (202) comprises a supply gas outlet (206) to be connected to a main gas supply line (210) of a docking station (400), and wherein said gas source (202) comprises a return gas inlet (208) to be connected to a main gas return line (212) of said docking station (400); wherein said gas source is as defined in any of the clauses 22-43.

Clause 77. Use of a docking station (400) according to any of the clauses 1-43 for incubation of a viable biological material.

Clause 78. Use of a modular incubator system (500) according to any of the clauses 44-75 for incubation of a viable biological material.

Clause 79. Use according to any of the clauses 77-78, wherein said biological material is being an oocyte or an embryo, such as a human oocyte or a human embryo.

Clause 80. A method of incubating a viable biological material, wherein said method comprises:
i) providing a modular incubator system (500) according to any of the clauses 44-75;
ii) providing a viable biological material;
iii) arranging said viable biological material in a culture dish (310) and subsequently arranging said culture dish in the interior (306) of a modular incubator chamber (300) of said modular incubator system (500);
iv) docking said modular incubator chamber (300) in a docking port (402) of said docking station (400) of said modular incubator system (500);
v) allowing said viable biological material to be incubated in said modular incubator chamber (300);
vi) via said gas supply system (200) of said docking station (400), supplying gas through the interior (306) of said modular incubator chamber (300) of said modular incubator system (500).

Clause 81. A method according to clause 80 further comprising the step of:
vi) removing said incubator chamber (300) from said docking port (402) of said docking station (400), when desired, in order to manually inspect the viable biological material, and optionally also to remove, add or exchange growth medium/media in said culture dish (310).

### LIST OF REFERENCE NUMERALS

2 First valve of valve system
4 Second valve of valve system
6 First valve body of first valve
8 First valve element of first valve
10 Front end of first valve body
12 Rear end of first valve body
14 First throughgoing channel of first valve
16 Second valve body of second valve
18 Second valve element of second valve
20 Front end of second valve
22 Rear end of second valve
24 Second throughgoing channel of second valve
26 First spring of first valve
28 Second spring of second valve
100 Valve system
200 Gas supply system
202 Gas source of gas supply system
204 Gas distribution system of gas supply system
206 Supply gas outlet of gas source
208 Return gas inlet of gas source
210 Main gas supply line of gas distribution system
212 Main gas return line of gas distribution system
214 Manifold pair
216 Inlet manifold of manifold pair
218 Outlet manifold of manifold pair
220 Inlet manifold connection point
222 Outlet manifold connection point
224 Supply line reference point
226 Return line reference point
228 Group of docking ports
228a Added group of docking ports
230 Inlet manifold connector of added inlet manifold
232 Outlet manifold connector of added outlet manifold
234 Connector of main gas supply line for adding new inlet manifold
236 Connector of gas return line for adding new inlet manifold
238 Shunt
240 Main line extension connector
242 Gas mixing box
244 Flow loop of gas supply system
246 Pump of gas source
247 Pump oscillation damper
248 Pressure sensor for sensing pressure of gas supplied to main gas supply line
249 Release valve
250 Inlet for N₂ gas
251 Inlet for CO₂ gas
252 N₂ valve
253 N₂ mass flow sensor
254 CO₂ valve
255 CO₂ mass flow sensor
256 Mass flow sensor for sensing the amount of return gas flowing into gas mixing box
258 O₂ sensor
260 CO₂ sensor
262 Temperature sensor
264 Pressure sensor
266 UV sanitizer
268 Filter
270 Gas mixing control system
300 Modular incubator chamber
302 Housing of modular incubator chamber
304 Lid of modular incubator chamber
306 Interior of modular incubator chamber
308 Culture dish support
310 Culture dish
312 Modular incubator chamber inlet opening for gas
314 Modular incubator chamber outlet opening for gas
316 Transparent window of housing of modular incubator chamber
318 Electric heating element
320 Electric power source
322 Electric connectors of modular incubator chamber
324 Display of housing of modular incubator chamber
326 Chamber engagement means of modular incubator chamber
340 First end of modular incubator chamber
342 Second end of modular incubator chamber
357 Bottom part of housing of modular incubator chamber
372 Light source
374 Thermostat
376 Thermostatic circuit
400 Docking station
402 Docking port of docking station
404 Docking port outlet opening for gas of docking port
406 Docking port inlet opening for gas of docking port
408 Image capturing device of docking port of docking station
410 Electric connector of docking port
414 Docking port engagement means of docking port of docking station
482 Displacement device for displacing image capturing unit
500 Modular incubator system
650 Control unit
652 Input device
654 Display unit
656 Data processing unit
658 Data storage
660 Image processing unit
D, D′, D″ Travel distance for gas
D1,D2 Partial travel distance for gas
D3,D4 Partial travel distance for gas
X Longitudinal direction of housing of modular incubator chamber
Y Transversal direction perpendicular to longitudinal direction of housing of modular incubator chamber

1-82. (canceled)

83. A docking station (400) for a modular incubator system (500), wherein said docking station comprises:
a number of docking ports (402) for receiving a modular incubator chamber (300); and
a gas supply system (200);
wherein in respect of one or more of said docking ports (402), preferably in respect of all said docking ports of said docking station (400), said docking port comprises a docking port outlet opening for gas (404) and a docking port inlet opening for gas (406);
wherein said gas supply system (200) comprises a gas source (202) and a gas distribution system (204);
wherein said gas source (202) comprises a supply gas outlet (206) and a return gas inlet (208);
wherein said gas distribution system (204) comprises a main gas supply line (210) and a main gas return line (212);
wherein said main gas supply line (210) of said gas distribution system (204) is being fluidly connected to said supply gas outlet (206) of said gas source (202) and wherein said main gas return line (212) is being fluidly connected to said return gas inlet (208) of said gas source (202);
wherein said gas distribution system (204) comprises a number of manifold pairs (214), wherein each manifold pair comprises an inlet manifold (216) and an outlet manifold (218);
wherein said inlet manifold (216) of each manifold pair (214) is fluidly connected to said main gas supply line (210) at an inlet manifold connection point (220);
wherein said outlet manifold (218) of each manifold pair (214) is fluidly connected to said main gas return line (212) at an outlet manifold connection point (222);
wherein a gas supply line reference point (224) arranged at a position upstream in relation to inlet manifold connection points (220) of all said inlet manifolds (216) is being defined on said main gas supply line (210);
wherein a gas return line reference point (226) arranged at a position downstream in relation to outlet manifold connection points (222) of all said outlet manifolds (218) is being defined on said main gas return line (212);
wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218);
wherein said gas distribution system (204) is designed in such a way that in respect of two or more docking ports (402) of said docking station, preferably in respect of all docking ports (402) of said docking station, the travel distance D for gas to travel from said gas supply line reference point (224) to said docking port (402) and from said docking port (402) to said gas return line reference point (226) upon conveying gas to and from said docking port (402) is being essentially equal; wherein the travel distance D, in respect of a specific docking port (402), is defined as:
D=D1+D2+D3+D4;
thereby ensuring an equal pressure drop through various modular incubator chambers (300) which are being docked in docking ports (402) of said docking station (400);
wherein D1 is defined as the distance from said gas supply line reference point (224) to the corresponding inlet manifold connection point (220) at said main gas supply line (210);
wherein D2 is defined as the distance from said corresponding manifold connection point

(220) at said main gas supply line (210) to said docking port outlet opening for gas (404);
wherein D3 is defined as the distance from said docking port inlet opening for gas (406) to the corresponding outlet manifold connection point (222) at said main gas return line (212);
wherein D4 is defined as the distance from said outlet manifold connection point (222) at said main gas return line (212) to said gas return line reference point (226).

84. A docking station (400) according to claim 83, wherein in respect of one or more of said docking ports (402), preferably in respect of all said docking ports, said docking port outlet opening for gas (404) comprises a valve (4), and said docking port inlet opening for gas (406) comprises a valve (4); wherein said valve (4) of said docking port outlet opening for gas (404) and said valve (4) of said docking port inlet opening for gas (406) each comprises a valve body (16) having a front end (20), a rear end (22) and a through-going channel (24) therein, and a spring-loaded displaceable valve element (18), wherein said displaceable valve element (18) is being arranged in said through-going channel (24); wherein said displaceable valve element (18) is being configured to be displaceable in said through-going channel (24) of said valve body (16) in such a way, that when not acted upon by an external force, said spring-loaded displaceable valve element

(18) is not being displaced in said through-going channel (24) of said valve body (16), thereby making said valve attain a closed configuration blocking passage of gas through said through-going channel (24), and in such a way, that when acted upon by an external force, said spring-loaded displaceable valve element (18) is being displaced in said through-going channel (24) of said valve body (16), thereby making said valve (4) attain an open configuration, allowing passage of gas through said through-going channel (24).

85. A docking station (400) according to claim 83, wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

86. A docking station (400) according to claim 85, wherein in respect of one or more specific docking ports (402) of said docking station (400), said specific docking port comprises its own dedicated image capturing device (408) which is configured to only capture images relating to a modular incubator chamber (300) which is being docked in said specific docking port (402); optionally wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482), such as an electrically driven and remotely controlled displacement device (482) for enabling displacement of said common image capturing device (408) in relation to said N adjacently arranged docking ports (402) of said docking station (400).

87. A docking station (400) according to claim 83, wherein said gas source (202) of said gas supply system (200) comprises a gas mixing box (242) comprising said supply gas outlet (206) and said return gas inlet (208) of said gas source, wherein said main gas supply line (210) of said gas distribution system (204) is being fluidly connected to said supply gas outlet (206), and wherein said main gas return line (212) of said gas distribution system (204) is being fluidly connected to said return gas inlet (208) of said gas source (202), thereby forming a flow loop (244) comprising said gas distribution system (204) and said gas mixing box (242); wherein said flow loop comprises a pump (246) for circulating gas in said loop.

88. A docking station (400) according to claim 87, wherein said gas source (202) comprises an O₂ sensor 258 for sensing the concentration of O₂ exiting said gas distribution system (204); and wherein said gas source (202) comprises a CO₂ sensor (260) for sensing the concentration of CO₂ exiting said gas distribution system (204), wherein said O₂ sensor and/or said CO₂ sensor optionally is/are being arranged downstream in relation to said pump (246).

89. A docking station (400) according to claim 87, wherein said gas source (202) comprises a gas mixing control system (270), wherein said gas mixing control system is electrically connected to one or more of the following sensors for receiving sensing signals therefrom: said N₂ mass flow sensor (253) for sensing the amount of N₂ flowing into said gas mixing box; said CO₂ mass flow sensor (255) for sensing the amount of CO₂ flowing into said gas mixing box; said mass flow sensor (256) for sensing the amount of return gas entering said gas mixing box; said O₂ sensor (258) for sensing the concentration of O₂ exiting said main gas return line (212) of said gas distribution system (204); said CO₂ sensor (260) for sensing the concentration of CO₂ exiting said main gas return line (212) of said gas distribution system (204); said temperature sensor (262) for sensing the temperature circulating in said flow loop (244); said pressure sensor (264) for sensing an absolute pressure in said flow loop (244), said pressure sensor (248) for sensing the pressure of gas supplied to said gas main gas supply line (210) of said distribution system (204).

90. A docking station (400) according to claim 89, wherein said gas mixing control system (270) is being configured to maintain a CO₂ concentration of gas entering said main gas supply line (210) of said gas distribution system (204) in the range of 5-10%, such as 6-9% or 7-8%; and/or an O₂ concentration of gas entering said main gas supply line (210) of said gas distribution system 204 in the range of 5-10%, such as 6-9% or 7-8%.

91. A modular incubator system (500) comprising a docking station (400) according to claim 83 in combination with one or more modular incubator chambers (300); wherein in respect of one or more of said one or more modular incubator chambers (300), said modular incubator chamber (300) comprises:
a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said modular incubator chamber (300) comprises a chamber inlet opening for gas (312) and a chamber outlet opening for gas (314), wherein said chamber inlet opening for gas (312) and said chamber outlet opening for gas (314) are being in fluid connection with the interior (306) of said modular incubator chamber.

92. A modular incubator system (500) according to claim 91, wherein in respect of one or more of said one or more modular incubator chambers (300), and in respect of one or more of said one or more docking ports (402) of said docking station (400), the position of said chamber inlet opening for gas (312) of said modular incubator chamber (300) and the position of said docking port outlet opening for gas (404) of said docking port (402) are adapted to each other in such a way that once docking said modular incubator chamber (300) in said docking port (402), said chamber inlet opening for gas (312) of said housing (302) of said modular incubator chamber

(300) and said docking port outlet opening for gas (404) of said docking port (402) will be in fluid connection; and in such a way that the position of said chamber outlet opening for gas (314) of said modular incubator chamber (300) and the position of said docking port inlet opening for gas (406) of said docking port (402) are adapted to each other in such a way that once docking said modular incubator chamber (300) in said docking port (402), said chamber outlet opening for gas (314) of said housing (302) of said modular incubator chamber (300) and said docking port inlet opening for gas (406) of said docking port (402) will be in fluid connection.

93. A modular incubator system (500) according to claim 91, wherein in respect of one or more of said one or more modular incubator chambers (300), said chamber inlet opening for gas (312) comprises a valve (2), and said chamber outlet opening for gas (314) comprises a valve (2); wherein said valve (2) of said chamber inlet opening for gas (312) and said valve (2) of said chamber outlet opening for gas (314) each comprises a valve body (6) having a front end (10), a rear end (12) and a through-going channel 14 therein, and a spring-loaded displaceable valve element (8), wherein said displaceable valve element (8) is being arranged in said through-going channel (14); wherein said displaceable valve element (8) is being configured to be displaceable in said through-going channel (14) of said valve body (6) in such a way, that when not acted upon by an external force, said spring-loaded displaceable valve element (8) is not being displaced in said through-going channel (14) of said valve body (6), thereby making said valve attain a closed configuration blocking passage of gas through said through-going channel (14), and in such a way, that when acted upon by an external force, said spring-loaded displaceable valve element (8) is being displaced in said through-going channel (14) of said valve body (6), thereby making said valve (2) attain an open configuration, allowing passage of gas through said through-going channel (14).

94. A modular incubator system (500) according to claim 93, wherein in respect of one or more of said one or more docking ports (402) of said docking station (400), said docking port(s) is/are as defined in claim 84, and wherein in respect of one or more of said one or more modular incubator chambers (300) said modular incubator chamber is as defined in claim 93, wherein said valves (2,4) are having dimensions and geometries in such a way that once docking said modular incubator chamber in said docking port (402) of said docking station (400), said displaceable valve element (8) of said valve (2) and said displaceable valve element (18) of said valve (4) will displace each other into their respective valve bodies (6,16), thereby opening said valves (2,4) of said docking port outlet opening for gas (404) and said chamber inlet opening for gas (312); and thereby opening said valves (2,4) of said chamber outlet opening for gas (314) and said docking port inlet opening for gas (406).

95. A modular incubator system (500) according to claim 91, wherein in respect of one or more of said one or more modular incubator chambers (300), said housing (302) of said modular incubator chamber (300) comprises a transparent window (316), and wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

96. A modular incubator system (500) according to claim 95, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

97. A modular incubator system (500) according to claim 91, wherein in respect of one or more of said one or more modular incubator chambers (300), said modular incubator chamber (300), in the interior (306) thereof, comprises a light source (372) for directing light to the area of the culture dish support (308) of said modular incubator chamber (300), thereby enabling illumination of a viable biological material in a situation of capturing images of said viable biological material.

98. A modular incubator system (500) according to claim 91, wherein said modular incubator system (500) comprises an image processing unit (660) for image processing of images captured by said image capturing device (408), wherein said modular incubator system (500) furthermore comprises a data storage (658) for storing images captured by said image capturing units 408 and/or for storing images processed by said image processing unit (660).

99. A modular incubator system 500 according to claim 91, wherein said modular incubator system comprises a control unit 650 for controlling the operation thereof.

100. A modular incubator system (500) according to claim 99, wherein said control unit (650) is being coupled to a data processing unit (656) and optionally also to a data storage (658) for aiding in handling information during controlling of said modular incubator system; wherein said control unit (650) optionally is being configured for effecting time lapse capturing of images by said image capturing device(s) (408).

101. A gas source (202) for providing gas to a docking station (400) according to claim 87, wherein said gas source (202) comprises a supply gas outlet (206) to be connected to a main gas supply line (210) of a docking station (400), and wherein said gas source (202) comprises a return gas inlet (208) to be connected to a main gas return line (212) of said docking station (400).

102. A method for incubation of a viable biological material by using of a docking station (400) according to claim 83.

103. A method of incubating a viable biological material according to claim 91, wherein said method comprises:
i) providing the modular incubator system (500);
ii) providing a viable biological material;
iii) arranging said viable biological material in a culture dish (310) and subsequently arranging said culture dish in the interior (306) of a modular incubator chamber (300) of said modular incubator system (500);
iv) docking said modular incubator chamber (300) in a docking port (402) of said docking station (400) of said modular incubator system (500);
v) allowing said viable biological material to be incubated in said modular incubator chamber (300);
vi) via said gas supply system (200) of said docking station (400), supplying gas through the interior (306) of said modular incubator chamber (300) of said modular incubator system (500).
